(19) United States
(12) Patent Application Publication
(10) Pub. No.: US 2022/0382246 A1
(43) Pub. Date: Dec. 1, 2022

(54) DIFFERENTIABLE SIMULATOR FOR ROBOTIC CUTTING

(71) Applicant: NVIDIA Corporation, Santa Clara, CA (US)

(72) Inventors: Eric Heiden, Los Angeles, CA (US); Fabio Tozeto Ramos, Seattle, WA (US); Yashraj Narang, Seattle, WA (US); Miles Macklin, Auckland (NZ); Dieter Fox, Seattle, WA (US); Animesh Garg, Toronto (CA); Mike Skolones, Santa Clara, CA (US)

(21) Appl. No.: 17/732,313

(22) Filed: Apr. 28, 2022

Related U.S. Application Data
(60) Provisional application No. 63/180,917, filed on Apr. 28, 2021.

(51) Int. Cl. G05B 19/4069; G06T 17/20; G05B 19/416; B25J 9/16

(52) U.S. Cl. CPC G05B 19/4069; G06T 17/205; G05B 19/4163; B25J 9/1664; B25J 9/1671

(57) ABSTRACT

A differentiable simulator for simulating the cutting of soft materials by a cutting instrument is provided. In accordance with one aspect of the disclosure, a method for simulating a cutting operation includes: receiving a mesh for an object, modifying the mesh to add virtual nodes associated with a predefined cutting plane, optimizing a set of parameters associated with a simulator based on ground-truth data, and running a simulation via the simulator to generate outputs that include trajectories associated with a cutting instrument. Optimizing the set of parameters can include performing inference based on a set of ground-truth trajectories captured using sensors to measure real-world cutting operations. The inference techniques can employ stochastic gradient descent, stochastic gradient Langevin dynamics, or a Bayesian approach. In an embodiment, the simulator can be utilized to generate control signals for a robot based on the simulated trajectories.

### CROSS-REFERENCE TO RELATED APPLICATIONS

This application claims the benefit of U.S. Provisional Application No. 63/180,917 titled “A DIFFERENTIABLE SIMULATOR FOR ROBOTIC CUTTING,” filed Apr. 28, 2021, the entire contents of which is incorporated herein by reference.

### BACKGROUND

Robotic cutting of soft materials is critical for applications such as food processing, household automation, and surgical manipulation. As in other areas of robotics, simulators can facilitate controller verification, policy learning, and dataset generation. Moreover, differentiable simulators can enable gradient-based optimization, which is invaluable for calibrating simulation parameters and optimizing controllers.

A simulation method referred to as a Finite Element Method (FEM) has been applied to simulate the continuum mechanics of biomaterial, which removes mesh elements as the knife or cutting object progresses through the object. However, this approach is not differentiable and requires the simulation parameters to be manually tuned to closely match the results to knife force profiles collected in the real-world. Remeshing algorithms have been developed to adapt the FEM such that crack propagation can be simulated at higher resolutions near the cutting interface. However, these methods are purely geometric and do not simulate the mechanics of cutting. An eXtended FEM (X-FEM) can simulate crack growth and cutting without remeshing, however this technique requires more computational resources, and no differentiable solution has been implemented at this time. ANSYS implements a Smoothed Particle Galerkin (SPG) method in LS-DYNA to simulate fracture and crack propagation. Again, the ANSYS solution is not differentiable and requires large computational resources.

Position-based dynamics and extended position-based dynamics have been used to implement interactive simulators for surgical cutting. However, these approaches are not as physically accurate as the FEM-based simulators and the simulations are highly dependent on the time step interval. Extensions to the Material Point Method (MPM) have been developed to simulate cutting. While one implementation proposes a particle-in-cell method that allows model material separation, this implementation is limited to a geometric operation that does not model the knife contact forces. Another implementation uses a continuous damage mechanics model, however this technique does not provide gradients for the simulation parameters to calibrate the simulator efficiently.

Purely machine-learning based approaches have also been developed. However, such deep learning models do not provide parameters that have real-world meaning and do not explicitly model the forces acting on the knife. Instead, latent feature vectors are inferred at each time step. In some learning-based approaches, force-torque measurements are incorporated into a learning-based controller, however these approaches also do not explicitly infer forces on the knife or cutting object or other quantities that can be used to infer physically meaningful simulation parameters. Thus, there is a need for addressing these issues and/or other issues associated with the prior art.

### SUMMARY

A differentiable simulator for simulating the cutting of soft materials by a cutting instrument is described herein. The simulator augments the finite element method (FEM) with a continuous contact model based on signed distance fields (SDF), as well as a continuous damage model that inserts springs on opposite sides of the cutting plane and allows them to weaken until zero stiffness to model crack formation. Differentiability brings a number of advantages for simulation technology. First, the simulator can be calibrated to match resultant forces and deformation fields from a state-of-the-art commercial solver and real-world cutting datasets, with generality across cutting velocities and object instances. Next, a Bayesian inference can be performed efficiently by leveraging the differentiability of the simulator, estimating posteriors over hundreds of parameters in a fraction of the time of derivative-free methods. Finally, the control parameters in the simulation can be optimized to minimize cutting forces via lateral slicing motions.

In accordance with a first aspect of the present disclosure, a method for simulating a cutting operation is disclosed. The method includes: receiving a mesh for an object, modifying the mesh to add virtual nodes associated with a predefined cutting plane, optimizing a set of parameters associated with a simulator based on ground-truth data, and running a simulation via the simulator to generate outputs that include trajectories associated with a cutting instrument.

In accordance with an embodiment of the first aspect, the set of parameters includes a set of spring constants for a plurality of virtual springs, each virtual spring associated with two virtual nodes.

In accordance with an embodiment of the first aspect, during the simulation, the set of spring constants are updated during a plurality of time steps based on contact forces calculated between the cutting instrument and the object.

In accordance with an embodiment of the first aspect, running the simulation includes, for each time step in a plurality of time steps: calculating external contact forces between the object and a surface due to gravity, calculating elastic forces for tetrahedral elements of the mesh based on a strain energy density formula, calculating contact forces between the object and the cutting instrument, updating the set of spring constants based on the contact forces, and calculating spring forces associated with the virtual springs.

In accordance with an embodiment of the first aspect, the contact forces include contact normal forces and friction forces derived based on a signed distance function (SDF) representation of the cutting instrument.

In accordance with an embodiment of the first aspect, the trajectories include an estimate of a force associated with the cutting instrument over a period of time.

In accordance with an embodiment of the first aspect, optimizing the set of parameters includes applying stochastic gradient descent based on a set of ground-truth trajectories captured using sensors to measure real-world cutting operations.

In accordance with an embodiment of the first aspect, optimizing the set of parameters includes applying an Adaptive Moment Estimation (Adam) optimizer to compute the set of parameters for the simulation.

In accordance with an embodiment of the first aspect, optimizing the set of parameters includes applying a stochastic gradient Langevin dynamics (SGLD) algorithm to compute the set of parameters for the simulation.

In accordance with an embodiment of the first aspect, optimizing the set of parameters includes applying a BayesSim algorithm to compute the set of parameters for the simulation.

In accordance with an embodiment of the first aspect, the method further includes generating control signals for a robot based on the outputs of the simulation.

In accordance with an embodiment of the first aspect, optimizing the set of parameters includes optimizing a trajectory of a cutting instrument using gradient-based descent and a Modified Differential Method of Multipliers (MDMM).

In accordance with a second aspect of the present disclosure, a system for simulating a cutting operation is disclosed. The system includes a memory storing a mesh for an object and a set of parameters associated with a simulator, and one or more processors coupled to the memory. The one or more processors are configured to: modify the mesh to add virtual nodes associated with a predefined cutting plane, optimize the set of parameters based on ground-truth data, and run a simulation via the simulator to generate outputs that include trajectories associated with a cutting instrument.

In accordance with an embodiment of the second aspect, running the simulation includes, for each time step in a plurality of time steps: calculating external contact forces between the object and a surface due to gravity, calculating elastic forces for tetrahedral elements of the mesh based on a strain energy density formula, calculating contact forces between the object and the cutting instrument, updating the set of spring constants based on the contact forces, and calculating spring forces associated with the virtual springs.

In accordance with an embodiment of the second aspect, the system further includes a robot. The one or more processors are further configured to generate control signals for the robot based on the outputs of the simulation.

In accordance with an embodiment of the second aspect, the trajectories include an estimate of a force associated with the cutting instrument over a period of time.

In accordance with an embodiment of the second aspect, optimizing the set of parameters comprises applying stochastic gradient descent based on a set of ground-truth trajectories captured using sensors to measure real-world cutting operations.

In accordance with a third aspect of the present disclosure, a non-transitory computer readable medium is disclosed. The computer-readable medium stores instructions that, responsive to being executed by one or more processors, cause the one or more processors to: receive a mesh for an object, modify the mesh to add virtual nodes associated with a predefined cutting plane, optimize a set of parameters associated with a simulator based on ground-truth data, and run a simulation via the simulator to generate outputs that include trajectories associated with a cutting instrument.

In accordance with an embodiment of the third aspect, running the simulation includes, for each time step in a plurality of time steps: calculating external contact forces between the object and a surface due to gravity, calculating elastic forces for tetrahedral elements of the mesh based on a strain energy density formula, calculating contact forces between the object and the cutting instrument, updating the set of spring constants based on the contact forces, and calculating spring forces associated with the virtual springs.

In accordance with an embodiment of the third aspect, the trajectories include an estimate of a force associated with the cutting instrument over a period of time.

### BRIEF DESCRIPTION OF THE DRAWINGS

The present systems and methods for a differentiable simulator for robotic cutting are described in detail below with reference to the attached drawing figures, wherein:

FIG. 1A depicts a flow chart of a method for simulating a cutting operation, in accordance with some embodiments.

FIG. 1B depicts a flow chart of a method for running a simulation, in accordance with some embodiments.

FIG. 2A illustrates a system capable of running the simulation, in accordance with some embodiments.

FIG. 2B illustrates a robotic system, in accordance with some embodiments.

FIG. 3 illustrates a signed distance function (SDF) representation of a cutting instrument, in accordance with some embodiments.

FIG. 4 illustrates an example parallel processing unit suitable for use in implementing some embodiments of the present disclosure.

FIG. 5A is a conceptual diagram of a processing system implemented using the PPU of FIG. 4, suitable for use in implementing some embodiments of the present disclosure.

FIG. 5B illustrates an exemplary system in which the various architecture and/or functionality of the various previous embodiments may be implemented.

FIG. 5C illustrates components of an exemplary system that can be used to train and utilize machine learning, in at least one embodiment.

### DETAILED DESCRIPTION

Systems and methods are disclosed related to a differentiable simulator for robotic cutting. The simulator is calibrated against ground-truth data from commercial solvers and real-world datasets. Calibration involves adjusting parameters of the simulator using, e.g., stochastic gradient descent based on ground-truth data. Once the simulator is calibrated, simulations for cutting objects can be executed to generate knife trajectories to optimize cutting forces within time constraints. The optimization generated by the simulator can be applied to a robot to cut a real-world object using a knife or other cutting instrument attached to the end-effector of the robot.

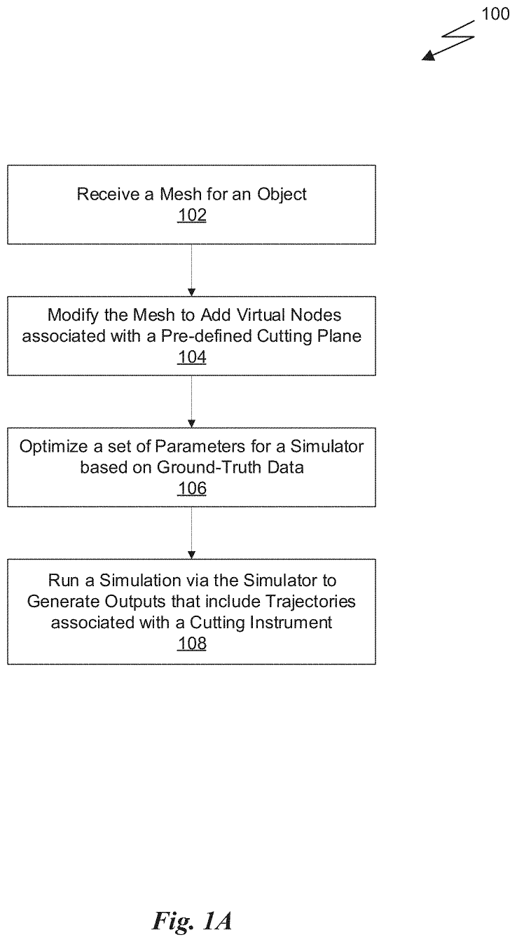

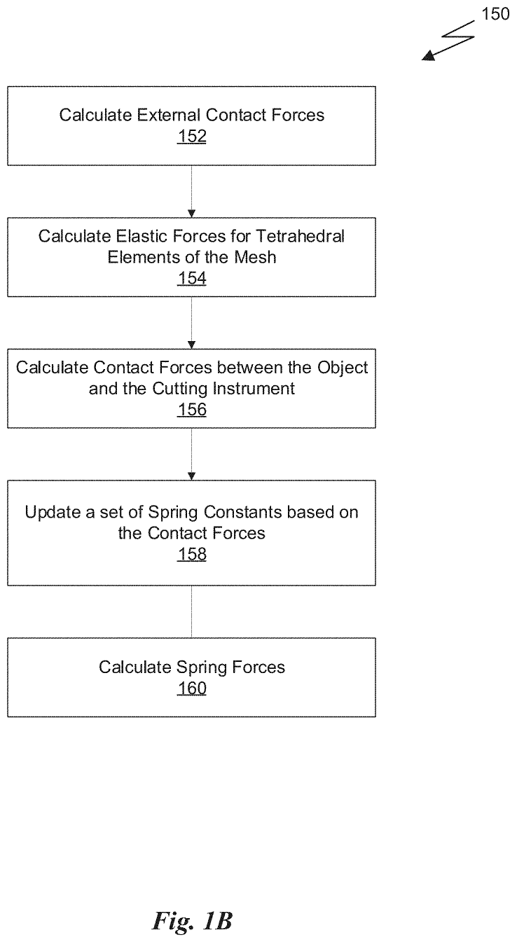

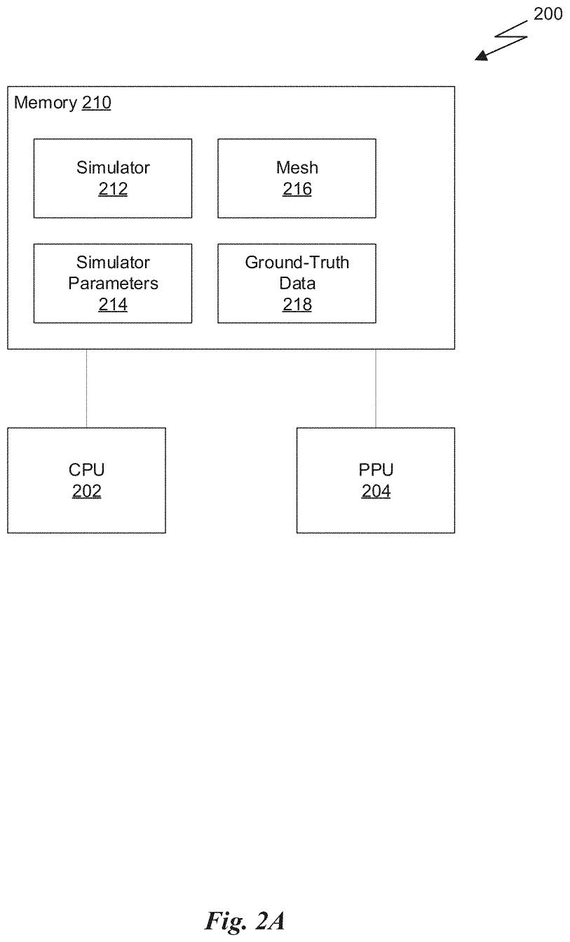

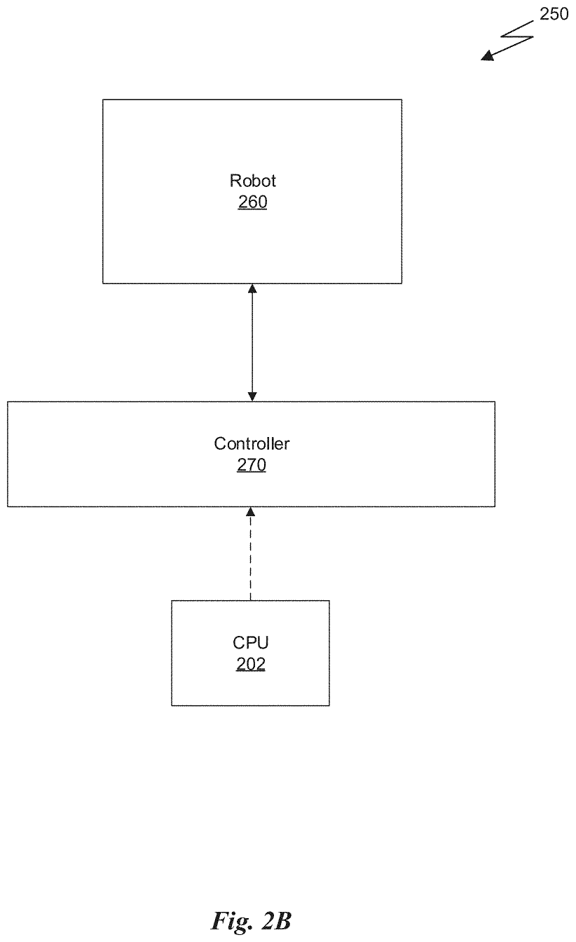

FIG. 1A depicts a flow chart of a method 100 for simulating a cutting operation, in accordance with some embodiments. Each block of method 100, described herein, comprises a computing process that may be performed using any combination of hardware, firmware, and/or software. For instance, various functions may be carried out by a processor executing instructions stored in memory. The method 100 may also be embodied as instructions stored on computer-readable media. The method 100 may be provided by a standalone application, a service or hosted service (standalone or in combination with another hosted service), or a plug-in to another product, to name a few potential embodiments. In addition, method 100 is described, by way of example, with respect to the system of FIG. 2. However, the method 100 may additionally or alternatively be executed by any one system, or any combination of systems, capable of executing the steps of the method 100 including, but not limited to, those systems described herein. Furthermore, persons of ordinary skill in the art will understand that any system that performs method 100 is within the scope and spirit of embodiments of the present disclosure.

The method 100 begins at step 102, where a mesh for an object is received. The object may be a geometric model of an object to be cut, such as a potato or an apple. The geometric model can include a series of points located on the surfaces of the object. In one embodiment, the mesh comprises a set of tetrahedral elements formed based on the points located on the surfaces of the object as well as other points generated inside the object. Each tetrahedral element includes four vertices of the model that form four triangles as the sides of the tetrahedron. Thus, an object may be represented by a set of tetrahedral elements that represent a volume of the object, where each tetrahedral element may share three vertices with an adjacent tetrahedral element.

At step 104, the mesh is modified to add virtual nodes associated with a cutting plane. In some embodiments, the mesh needs to be adapted to simulate the cutting operation because the mesh, as received, may not define points along the cutting plane in order to simulate the cutting operation. Thus, virtual nodes can be added to the mesh such that the tetrahedral elements in the mesh will align with the pre-defined cutting plane for purposes of simulating the cutting operation. Each point along the cutting plane can be modeled as a pair of virtual nodes connected by a virtual spring element. As the cutting instrument forces the virtual nodes apart, the virtual spring element can be used to calculate a force that represents a resistance of the object to separate at the virtual nodes, which is translated into a force applied to the cutting instrument.

In an embodiment, each tetrahedral element of the mesh that intersects the cutting plane is duplicated such that one tetrahedral element of the pair of tetrahedral elements is associated with a first side of the cutting plane and another tetrahedral element of the pair of tetrahedral elements is associated with a second side of the cutting plane. Virtual nodes located on the edges of the tetrahedral element at a location that intersects the cutting plane are added to each pair of tetrahedral elements and then virtual spring elements are connected to the virtual nodes. The virtual spring elements allow the simulator to simulate damage occurring during the cutting process in an entirely continuous (and thereby differentiable) manner, by weakening their stiffness values (i.e., spring constants) as knife contact forces are applied over time.

During the simulation, one tetrahedral element of each pair will move in opposite directions away from the cutting plane to simulate the deformation and fracturing of the object in response to the cutting instrument moving through the object. Each virtual node's position \tilde{x} and velocity 13 is defined by the coordinates of its two parent vertices, indexed as i and j:

\tilde{x}=(1−u)xᵢ+uxⱼ (Eq. 1)

\tilde{v}(1−u)vᵢ+uvⱼ (Eq. 2)

The edge sections along the non-empty portions of the duplicated mesh elements participate in contact dynamics and propagate the resulting contact forces back to their parent nodes.

It will be appreciated that the mesh may be modified based on an assumption that the entire cutting plane is predefined before the simulation begins, and therefore step 104 is performed once during (i.e., prior to) the simulation. However, in other embodiments, the simulator can be configured to modify the mesh during each iteration timestep of the simulation. This can allow for the cutting plane to change dynamically (e.g., as the knife position is changed to simulate complex cutting motions) such that the mesh augmentation step is interweaved with the actual cutting simulation.

At step 106, a set of parameters for the simulator are optimized based on ground-truth data. The ground-truth data can include trajectories of real-world cutting operations that are captured using sensors attached to a cutting instrument. For example, force sensors (e.g., strain gauges, piezoelectric elements, etc.) can be used to measure the force required by a robot to cut through an object. The trajectories include a trace of the force measurement over a period of time during the cutting operation. In other words, a trajectory is a dynamic measurement of force throughout the cutting operation and not a scalar measurement of, e.g., a peak force applied to the cutting instrument.

In some embodiments, the set of parameters include, e.g., spring constants kₑ for each of the virtual spring elements. The spring constants are used to calculate a spring force based on Hooke's law:

F=−kx (Eq. 3)

The set of parameters can also include: geometrical parameters of the cutting instrument such as a radius of the knife edge, a height of the knife, a length of the knife, and a radius of the knife spine; initial position and velocity of the cutting instrument; a spring damping coefficient; dynamic parameters for the cutting instrument such as a stiffness of the cutting instrument, a friction coefficient of the cutting instrument, a radius around the signed distance function (SDF) to consider for contact dynamic, and/or material properties such as Young's modulus, Poisson's ratio, and density of the object. This listing of parameters is not exclusive and other parameters can be included in the model. Further, some parameters can be represented as a distribution. For example, a spring constant for each virtual spring may be randomly selected during execution of a simulation based on a mean and standard deviation of the spring constant parameter such that each virtual spring element may have different initial values for the spring constant. This can represent an object that is not exactly homogenous internally.

In one embodiment, the set of parameters are optimized using stochastic gradient descent. The contact model dynamics used by the simulator can be differentiable such that the parameters can be optimized by comparing outputs of the simulation with ground-truth data and then adjusting the set of parameters based on gradients for the parameters. In an embodiment, the parameters are optimized using an Adaptive Moment Estimation (ADAM) optimizer. In another embodiment, the posterior distribution over parameters are optimized using stochastic gradient Langevin dynamics (SGLD).

At step 108, a simulation is run using the simulator to generate outputs that include trajectories associated with a cutting instrument. The trajectories represent forces on the cutting instrument over a period of time as estimated by the simulator over a series of time steps.

FIG. 1B depicts a flow chart of a method 150 for running a simulation, in accordance with some embodiments. In an embodiment, the method 150 can be implemented at step 108 of method 100. The method 150 may be executed by any one system, or any combination of systems, capable of executing the steps of the method 150 including, but not limited to, those systems described herein. Furthermore, persons of ordinary skill in the art will understand that any system that performs method 150 is within the scope and spirit of embodiments of the present disclosure.

At step 152, the simulator calculates external contact forces between the object and a cutting surface. The cutting surface can represent a cutting board, the ground, or the like. In some embodiments, the set of parameters of the simulator can include parameters related to the cutting surface, such as an elastic modulus, stiffness, or the like. The external contact forces can include forces due to gravity acting on the object forces between the cutting surface and the points of contact with the object.

In an embodiment, contact forces between the mesh of the object and the cutting surface are calculated between the mesh vertices of the object, each represented as a sphere, and a half-space for the cutting surface. To prevent the object from sliding off the cutting surface during the simulation, boundary conditions may be applied to the mesh vertices that touch the table and/or are within a threshold distance (e.g., 1 cm) of the cutting surface.

At step 154, the simulator calculates elastic forces for each tetrahedral element of the mesh. The elastic forces can be calculated based on a strain-energy density function modeled by the following:

$$Ψ = \frac{μ}{2} ( {I}_{C} - 3 ) + \frac{λ}{2} {( J - α )}^{2} - \frac{μ}{2} log ( {I}_{C} + 1 ) , \qquad ( Eq . 4 )$$

where λ, μ are the Lamè parameters and α is a constant. J=det(F) is the relative volume change, I_{C}=tr(F^{T} F) is the first Cauchy invariant of strain, and F is the deformation gradient of the tetrahedral element.

At step 156, the simulator calculates contact forces between the cutting instrument and the object. At each time step in the simulation, the position of the cutting instrument can be represented by a signed distance function (SDF). The SDF is used to find the closest point on each edge of the tetrahedral element to the cutting instrument. Once this point is found, a penetration depth and contact normal can be found by querying the SDF and the gradient of the SDF, which can then be used to calculate contact normal forces and friction forces from the cutting instrument against the tetrahedral element.

A contact model is implemented that represents a shape of the cutting instrument by the SDF. In an embodiment, a number of iterations (e.g., 20 iterations) of a Frank-Wolfe algorithm (illustrated in Table 1) are run that uses gradient information from the SDF to find a locally optimal solution for a barycentric coordinate u∈[0, 1] on an edge (p₁, p₂) of the tetrahedral elements with the smallest distance with respect to the SDF.

TABLE 1

```
u←0.5
for i = 0 . . . max_iterations do
  $δ ← \frac{δ}{δ u} ϕ ( ( 1 - u ) {p}_{1} + {up}_{2} )$
  if δ < 0 then
    s←l
  else
    s←0
  end
  $γ ← \frac{2}{2 + i}$
  u ← u + γ (s − u)
end
```

Using the point associated with the calculated barycentric coordinate, the simulator can calculate the penetration depth and contact normal by querying the SDF and its gradient. These quantities, combined with a relative velocity between the cutting instrument and the mesh vertices, are used to calculate contact normal forces between the cutting instrument and the vertices of the mesh, as well as friction forces following a continuous friction model. In an embodiment, the contact normal forces between the cutting instrument and the vertices of the mesh are calculated in accordance with a technique illustrated in Brown, P., “Contact modeling for forward dynamics of human motion,” Thesis, University of Waterloo (2017), which is incorporated by reference herein in its entirety.

At step 158, a set of spring constants are updated based on the contact forces. The simulator model represents damage from the cutting instrument by reducing the stiffness of the spring linearly based on the force applied by the cutting instrument until the spring constant representing the stiffness of the spring is reduced to zero. When the spring constant is reduced to zero, this represents the cutting instrument successfully separating the two sides of the object on opposite sides of the cutting plane. Thus, for each time step of the simulation, the spring constants for each virtual spring element can be reduced in accordance with the contact forces calculated at that node (i.e., for the tetrahedral element sharing a vertices with one of the virtual nodes associated with that virtual spring element).

Damage refers to a macroscopic reduction in stiffness or strength of a material caused by the formation and growth of microscopic defects (e.g., voids and microcracks). For fruits and vegetables, which often have limited plastic deformation (such as with plastic or metal objects), damage can be approximated by a reduction in the elastic modulus of the material, or in a discrete mesh-based formulation, in the components of the stiffness matrix (i.e., the spring constants associated with the set of spring elements). As the cutting instrument applies force to the cutting interface, the stiffness of the springs that are in contact with the knife are linearly decreased as given by the following equation:

k′ₑ=kₑ−γ∥f_{knife}∥, (Eq. 5)=

where f_{knife} is the contact force calculated between the cutting instrument and the edge of a tetrahedral element (i.e., the force applied to the virtual spring element between two virtual nodes associated with the edge), kₑ is the initial spring constant for the spring, and γ∈[0, 1] is a coefficient that controls the “softness” of the spring (i.e., how easily the material weakens and separates as the force is applied by the cutting instrument).

At step 160, spring forces are calculated for each virtual spring element. In an embodiment, the spring forces are calculated based on a displacement between the virtual nodes attached to each spring element and the corresponding spring constant for the spring element.

The simulator can repeat method 150 for a number of iterations of a simulation loop in order to calculate the location and velocity of mesh elements and the cutting instrument at each time step of the simulation. An algorithm implemented by the simulator is shown in Table 2:

```
TABLE 2
for i= 1... timesteps do
  Compute gravity and external contact forces fₑₓₜ between object mesh
    and cutting surface
  Compute elastic forces f_{elastic} following a constitutive model given in
  Equation 4
  Compute contact forces f_{knife} using the SDF
  Update spring element stiffness as given in Equation 5
  Compute spring forces f_{spring}
  Semi-implicit Euler integration of particles:
    v^{t+1} ← v^{t} + ΔtM⁻¹(f_{knife} + f_{spring} + f_{elastic} + fₑₓₜ)
    x^{t+1} ← x^{t} + Δtv^{t+1}
  Euler integration of knife velocity
end
```

In other words, for each time step of the simulation, the forces are first calculated using the methods set forth above and then the location and velocities of the mesh vertices (i.e., particles) are calculated along with an updated position of the cutting instrument based on the simulated cutting instrument velocity. In an embodiment, a suitable time step for the simulation is set as Δt=10⁻⁵s, although other suitable time steps can be used to increase or decrease the number of iterations performed over the simulation.

A trajectory of the cutting instrument can be optimized as part of the simulation. In particular, an optimized trajectory of the cutting instrument may be calculated and utilized as part of step 156 when determining contact normal forces between the cutting instrument and the vertices of the mesh. In an embodiment, a trajectory of the cutting instrument can be represented by k equidistant keyframes in time (e.g., k=5). Three parameters are optimized per keyframe, including an amplitude of the lateral (e.g., along a z-axis) sinusoidal velocity, aᵢ, a frequency of the lateral sinusoidal velocity, bᵢ, and a vertical (e.g., along a y-axis) velocity, cᵢ.

To allow for a smooth interpolation between the keyframes, and a propagation of gradients from all trajectory parameters at each time step of the simulation, the simulator can weight the contribution of all keyframe parameters on the entire trajectory via a radial basis function (RBF) kernel. The RBF kernel uses a squared norm of the difference between a current time t difference and a predefined keyframe times to compute the weight contribution w∈ [symbol] ^{k} of the keyframe parameters:

$$w ( t ) = exp ( - \frac{{‖ t - w ‖}^{2}}{2 {σ}^{2}} ) \qquad ( Eq . 6 )$$

In effect, a non-zero contribution on the trajectory is maintained from all keyframes at all times, which eases gradient-based optimization. The kernel width, σ, controls how smoothed out the contributions of the keyframe parameters become. In an embodiment, through experimentation, a value of σ=\sqrt{0.03} was shown to be an appropriate choice, although other values may be chosen for a different application.

To compute a horizontal velocity, ż_{knife}(t), and a vertical velocity, \dot{y}_{knife}(t), of the cutting instrument at time t=[0 . . . T], the keyframe parameters in vector form a, b, c∈ [symbol] ^{k} are combined with the time-dependent weighting contribution from Equation 6:

ż_{knife}(t)=a·w(t)cos(b·w(t)t) (Eq. 7)

\dot{y}_{knife}(t)=c·w(t) (Eq. 8)

The mean force of the cutting instrument plus the vertical velocity is minimized over the entire length of the trajectory. Thus, high actuation effort by the robot controlling the cutting instrument is penalized while still maintaining fast progression of the cutting process:

$$\begin{matrix}minimize \\ u = [ a , b , c , ]\end{matrix} ℒ = \frac{1}{T} \int f ( t , a , b , c ) + {\dot{y}}_{knife} ( t ) dt \qquad ( Eq . 9 )$$

$$\vert {z}_{knife} ( t ) \vert \leq \frac{1}{2} {l}_{knife} , \qquad ( Eq . 10 )$$

where T is the time at the end of the trajectory, f(t, a, b, c) is the simulation step that returns the cutting instrument force norm ∥f_{knife}∥ at time step t given the trajectory parameters u=[a, b, c], and l_{knife} is the length of the cutting instrument. A hard constraint is imposed by Equation 10 to ensure that the lateral trajectory does not move too far along the length of the cutting instrument (which would trivially minimize the force from the cutting instrument).

Constrained optimization problems are typically solved by converting them to unconstrained optimization problems through the introduction of Lagrange multipliers. However, the critical points to such Lagrangians often tend to be saddle points, which gradient-descent-style algorithms, such as Adam, will not converge toward. In order to make the unconstrained optimization problem amenable to gradient-descent, following a modified differential method of multipliers (MDMM), the simulator may introduce a penalty term for the trajectory parameters:

$${E}_{penalty} = \frac{c}{2} {( g ( u ) )}^{2} , \qquad ( Eq . 11 )$$

which acts as an attractor to the energy function being optimized for, where c is a damping factor and g(u) is an equality constraint. To include the inequality constraint in Equation 10, a slack variable γ∈ [symbol] that becomes part of u is introduced:

$$g ( u ) = \vert {z}_{knife} ( t ) \vert - \frac{1}{2} {l}_{knife} - {γ}^{2} , \qquad ( Eq . 12 )$$

The update rule for the trajectory parameters is then given as follows:

$${u}^{′} = u - \frac{\partial ℒ}{\partial u} - λ \frac{\partial g}{\partial u} - cg ( u ) \frac{\partial g}{\partial u} , \qquad ( Eq . 13 )$$

$${λ}^{′} = λ + g ( u ) , \qquad ( Eq . 14 )$$

Using gradient-based trajectory optimization, an intuitive cutting strategy emerges. The simulator defines the cost function in Equation 9 to penalize the mean cutting instrument force and inverse velocity, and the simulator parameterizes the trajectory via keyframes in time that define a downward velocity and sinusoidal time-varying horizontal velocity, which is similar to common knife cutting motions in the real-world which use back and force motion to reduce the downward force used to cut an object.

More illustrative information will now be set forth regarding various optional architectures and features with which the foregoing framework may be implemented, per the desires of the user. It should be strongly noted that the following information is set forth for illustrative purposes and should not be construed as limiting in any manner. Any of the following features may be optionally incorporated with or without the exclusion of other features described.

FIG. 2A illustrates a system 200 capable of running the simulation, in accordance with some embodiments. The system 200 includes a memory 210 and one or more processors. As depicted in FIG. 2A, the processors can include a central processing unit (CPU) 202 and/or a parallel processing unit (PPU) 204. The memory 210 includes a simulator 212, a set of parameters 214, a mesh 216, and ground-truth data 218.

The simulator 210 is a set of instructions that, when executed by the CPU 202 and/or the PPU 204, perform a series of operations to run a simulation of a cutting operation on an object represented by the mesh 216. The instructions implement a set of algorithms for calculating trajectories associated with the cutting instrument.

In an embodiment, the ground-truth data 218 includes trajectories measured using sensors in real-world cutting operations. Alternatively, the ground-truth data 218 can also include simulation results from other types of simulators that, for example, may be much more computationally intensive compared to the simulator 210. While such complex simulators may not be sufficient for use in, for example, robotic control (e.g., because the simulation may take too long to be useful in calculating control signals for a robot), the outputs of these simulations can be used to tune the parameters of the simulator 210. To be clear, complex simulators that are not suitable for robotic control can be used to train the simulator 210, which may be suitable for robotic control.

While not shown explicitly, the system 200 can also include a robotic interface that is coupled to a robot. The processor(s) can be configured to generate control signals for the robot based on, e.g., the outputs generated by a simulation of a cutting operation.

FIG. 2B illustrates a robotic system 250, in accordance with some embodiments.

The robotic system 250 includes a robot 260 and a controller 270. The controller 270 generates control signals for the robot 260. The control signals can, e.g., cause one or more actuators to effect a motion of a cutting instrument at the end of a robot arm. In an embodiment, the control signals may be generated based on the outputs of the simulation. For example, the CPU 202 and/or PPU 204 may generate trajectories that are transmitted to the controller 270, which uses the trajectories to calculate control signals to match the trajectories.

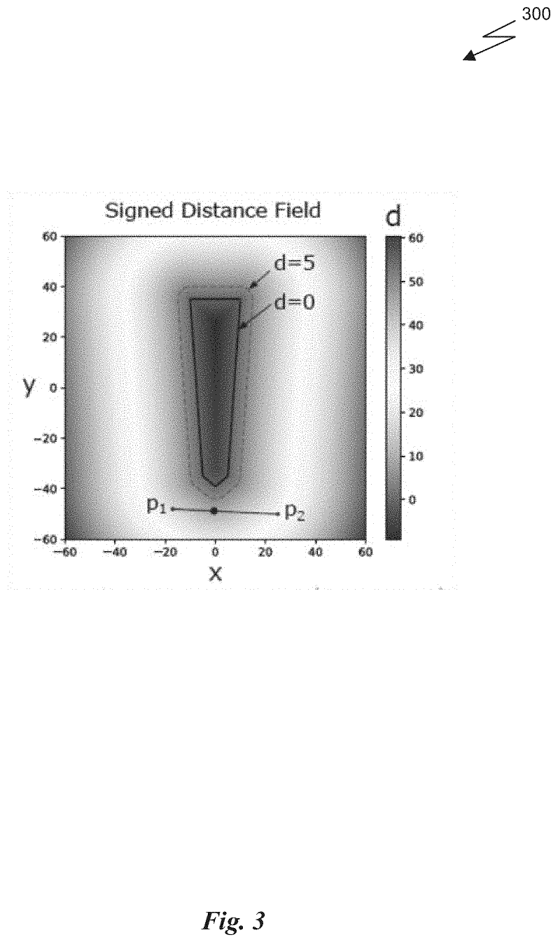

FIG. 3 illustrates a signed distance function (SDF) representation of a cutting instrument, in accordance with some embodiments. The SDF is a field where values in the field represent a closest distance from that point to the surface of the cutting instrument.

As shown in FIG. 3, an edge of a tetrahedral element is given as (p₁, p₂) and a profile of a cutting instrument is shown as a line at d=0. A dashed line at d=5 represents a set of points that are equidistant from the closest point on the profile of the cutting instrument. It will be appreciated that the SDF becomes more rounded as distances from the profile increase. The barycentric coordinate u is equal to zero (0) at p₁ and one (1) at p₂, and the closest point along the edge is given as the minimum value of the SDF along the line from p₁ to p₂. Again, this value can be estimated using the Frank-Wolfe algorithm illustrated in Table 1.

While it may be possible to manually tune parameters of low-dimensional, phenomenological models, the task for identifying the simulation parameters of the simulator 210 is daunting. In order to calibrate the simulator 210, automatic differentiation and acceleration using a PPU may be leveraged to compute gradients for all parameters of the simulator 210. Moreover, Bayesian inference methods can be used to estimate posterior distributions for the high-dimensional parameter set given physical observations (i.e., measured results) such as the force profile of the cutting instrument. The result is a simulator with the capacity to identify its own uncertainty about the physical world, leading to more robust simulation.

In an embodiment, the set of simulation parameters can be defined as θ, and the distribution over simulation parameters p(θ) is a uniform distribution. The likelihood function given by a set of real-world observations of the simulation parameters Or is defined as:

p(ϕ^{r}|θ)=exp{−∥ϕ_{θ}^{s}−ϕ^{r}∥_{L}}, (Eq. 15)

where ϕ_{θ}^{s} is an equivalent simulated trajectory, and ∥·∥_{L} is the L-norm. In an embodiment, the L1 norm is used in Equation 15. The posterior can then be computed as:

$$p ( θ \vert {ϕ}^{r} ) = \frac{1}{Z} p ( {ϕ}^{r} \vert θ ) p ( θ ) , \qquad ( Eq . 16 )$$

where Z is a normalizing constant. In various embodiments, different techniques for inferring simulator parameters may be implemented. In a first approach, a solution that uses stochastic gradient descent is employed. In a second approach, a stochastic gradient Langevin dynamics (SGLD) method is employed. In a third approach, a likelihood free approach is employed.

In accordance with an embodiment, simulation parameters are inferred using a stochastic gradient descent solution that uses an Adaptive Moment Estimation (Adam) optimizer, which is a first-order technique that scales the parameter gradients with respect to their running averages and variances. The Adam optimizer is fully deterministic and will attempt to find a locally optimal point estimate to the parameters that minimizes the estimation loss. More specifically, the loss is defined as:

l(θ)=log p(ϕ^{r}|θ), (Eq. 17)

and gradients are computed with respect to the simulator parameters θ that minimize the loss between a real trajectory ϕ^{r} and the simulated trajectories ϕ_{θ}^{s}.

At its core, the Adam optimize updates the parameters θ iterively as θᵢ←θᵢ₋₁−α·\hat{m}ᵢ/(\sqrt{vᵢ}+ε), where α is the learning rate, \hat{m}ᵢ and \hat{v}ᵢ represent the first and second order decaying averages after correcting for biases, and ε is a small value to prevent numerical issues. The Adam optimizer algorithm is illustrated in Table 3:

TABLE 3

```
m₀ ← 0
v₀ ← 0
for i = 1... max_iterations do
  gᵢ ← ∇_{θ}l(θᵢ₋₁)
  mᵢ ← β₁ · mᵢ₋₁ + (1 − β₁) · gᵢ
  vᵢ ← β₂ · vᵢ₋₁ + (1 − β₂) · gᵢ²
  \hat{m}ᵢ ← mᵢ/(1 − β₁ⁱ).
  \hat{v}ᵢ ← vᵢ/(1 − β₂ⁱ)
  θᵢ ← θᵢ₋₁ − α · \hat{m}ᵢ/ [symbol]    + ε)
end
return θᵢ
```

In accordance with another embodiment, SGLD is used as an alternative to the Adam optimizer. SGLD combines the benefits of having access to parameter gradients with well-established sampling-based methods for probabilistic inference to significantly scale the parameter set to high dimensions at a tractable computational cost. SGLD can be viewed as an iterative stochastic gradient optimization approach with the addition of Gaussian noise at every iteration, after scaling the noise by a pre-conditioner factor.

Given a sequence of trajectories generated by the simulator and a sequence of observed trajectories Φ={ϕᵢ^{s}, ϕᵢ^{r}}ᵢ₌₁^{N}, a posterior distribution can be defined as:

p(θ|Φ)∝p(θ)Πᵢ₌₁^{N}p(ϕᵢ^{r}|θ), (Eq. 18)

SGLD takes the energy function of the posterior denoted by

$$U ( θ ) = - {Σ}_{i = 1}^{N} \frac{1}{N} log p ( Φ \vert θ ) - log p ( θ )$$

to sample from the posterior using the following rule:

$${θ}_{t + 1} = {θ}_{t} - \frac{α}{2} A ( {θ}_{t} ) ∇ U ( {θ}_{t} ) + {η}_{t} , \qquad ( Eq . 19 )$$

where ηₜ [symbol] (0, A(θ_{t)α), α is the learning rate, and A is a pre-conditioner. After an initial burning in phase necessary for the Markov chain to converge, m samples can be stored to recover an approximate posterior given by}

$$p ( θ \vert Φ ) \approx \frac{1}{m} {Σ}_{i = 1}^{m} δ ( {θ}_{i} ) .$$

The choice of a proper pre-conditioner A is important to the performance of this algorithm. In an embodiment, the pre-conditioner is computed as an approximation of a Fisher information matrix of the posterior distribution given by A. This approximation can then be sequentially updated using the gradient of the energy function. Specifically, the pre-conditioner A∈ [symbol] ^{m×m} and momentum V∈ [symbol] ^{m} are updated as follows:

V(θₜ)=βV(θₜ₋₁)+(1−β)∇U(θ)⊙∇U(θₜ), (Eq. 20)

A(θₜ)=diag(1Ø(ε+\sqrt{V(θₜ)}), (Eq. 21)

where ε>0 is a small diagonal bias (e.g., ε=10⁻⁸) added to the pre-conditioner to prevent degeneration, and β(e.g., 0.95) is the exponential decay rate of the pre-conditioner. ⊙ and Ø are element-wise multiplication and division operators, respectively. The SGLD algorithm is illustrated below in Table 4:

TABLE 4

```
m₀ ← 0
v₀ ← 0
for i = 1... max_iterations do
  gᵢ ← ∇_{θ}U(θᵢ₋₁)
  mᵢ ← β₁ · mᵢ₋₁ + (1 − β₁) · gᵢ
  vᵢ ← β₂ · vᵢ₋₁ + (1 − β₂) · gᵢ²
  \hat{m}ᵢ ← mᵢ/(1 − β₁ⁱ).
  \hat{v}ᵢ ← vᵢ/(1 − β₂ⁱ)
  Aᵢ ← diag (1 [symbol]     ( [symbol]    + ε)
  ηᵢ~ [symbol]   (0, αAᵢ)
  θᵢ ← θᵢ₋₁ − α · \hat{m}ᵢ · Aᵢ + ηᵢ
end
return θᵢ
```

In yet another embodiment, as an alternative to the Adam optimizer or the SGLD technique, BayesSim is employed to estimate the parameters of a black-box simulator. Such simulators are generally not differentiable, but can generate trajectories by computing a forward model. The technique includes learning a conditional density q(θ|ϕ), which may be represented as a Gaussian Mixture Model (GMM), where θ are the simulation parameters and ϕ are trajectories or summary statistics of trajectories.

BayesSim approximates the posterior over the simulation parameters as:

p(θ|ϕ)≈p(θ)/\tilde{p}(θ)q(θ|ϕ), (Eq. 22)

where p(θ) is the prior distribution and \tilde{p}(θ) is a propositional prior used to sample the simulator and collect N samples, {θᵢ, ϕᵢ^{s}}_{i—1}^{N}, to learn q(θ|ϕ). When a real trajectory ϕ^{r} is observed, BayesSim computes p(θ|ϕ=ϕ^{r}) that represents the posterior over simulation parameters given the real data.

For the BayesSim baseline, a mixture density network (MDN) is trained representing q(θ|ϕ) with a number (e.g., 10) components on a dataset consisting of a plurality of trajectories (e.g., 500) that have been generated in the simulator by uniformly sampling the simulation parameters within their respective bounds. In an embodiment, only a subset of the parameters may be sampled to limit the exponential increase in sample complexity. Summary statistics can be calculated by down-sampling the profiles consisting of, e.g., thousands of time step iterations to create a summary downsampled by a factor of 1000 using polyphaser filtering.

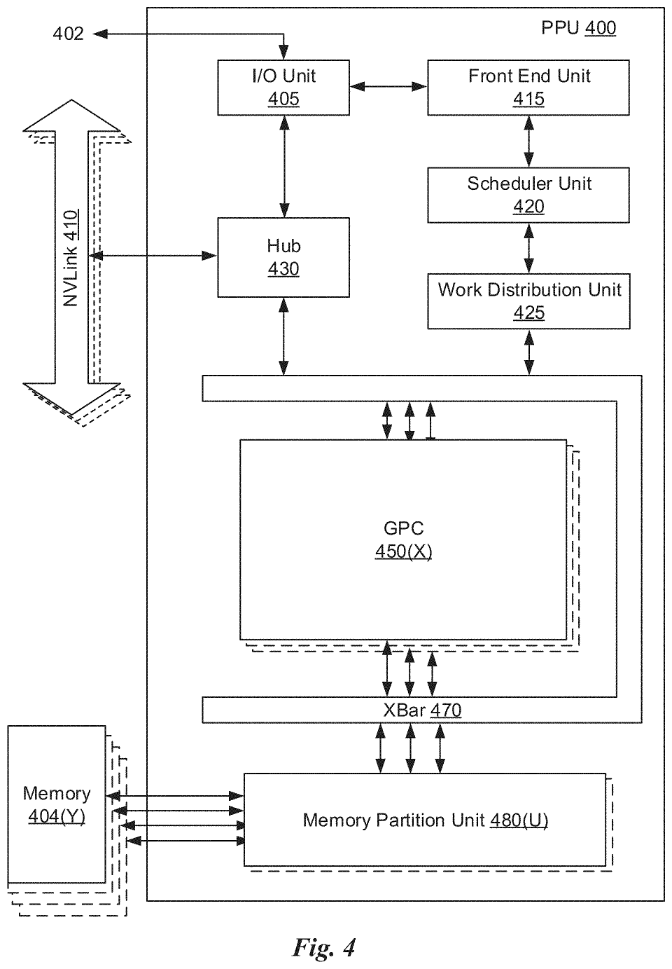

The simulator can be implemented by one or more processors, including a central processing unit (CPU) and/or a parallel processing unit (PPU), such as the PPU 400 depicted in FIG. 4. However, one of skill in the art will recognize that any system configured to execute the operations of the simulator is within the scope of the present disclosure. In some embodiments, the simulator may be implemented as a service accessible via a network, such as the Internet. A client device can send instructions to the service to run one or more simulations.

Parallel Processing Architecture

FIG. 4 illustrates a parallel processing unit (PPU) 400, in accordance with an embodiment. In an embodiment, a processor such as the PPU 400 may be configured to implement a neural network model. The neural network model may be implemented as software instructions executed by the processor or, in other embodiments, the processor can include a matrix of hardware elements configured to process a set of inputs (e.g., electrical signals representing values) to generate a set of outputs, which can represent activations of the neural network model. In yet other embodiments, the neural network model can be implemented as a combination of software instructions and processing performed by a matrix of hardware elements. Implementing the neural network model can include determining a set of parameters for the neural network model through, e.g., supervised or unsupervised training of the neural network model as well as, or in the alternative, performing inference using the set of parameters to process novel sets of inputs.

In an embodiment, the PPU 400 is a multi-threaded processor that is implemented on one or more integrated circuit devices. The PPU 400 is a latency hiding architecture designed to process many threads in parallel. A thread (e.g., a thread of execution) is an instantiation of a set of instructions configured to be executed by the PPU 400. In an embodiment, the PPU 400 is a graphics processing unit (GPU) configured to implement a graphics rendering pipeline for processing three-dimensional (3D) graphics data in order to generate two-dimensional (2D) image data for display on a display device. In other embodiments, the PPU 400 may be utilized for performing general-purpose computations. While one exemplary parallel processor is provided herein for illustrative purposes, it should be strongly noted that such processor is set forth for illustrative purposes only, and that any processor may be employed to supplement and/or substitute for the same.

One or more PPUs 400 may be configured to accelerate thousands of High Performance Computing (HPC), data center, cloud computing, and machine learning applications. The PPU 400 may be configured to accelerate numerous deep learning systems and applications for autonomous vehicles, simulation, computational graphics such as ray or path tracing, deep learning, high-accuracy speech, image, and text recognition systems, intelligent video analytics, molecular simulations, drug discovery, disease diagnosis, weather forecasting, big data analytics, astronomy, molecular dynamics simulation, financial modeling, robotics, factory automation, real-time language translation, online search optimizations, and personalized user recommendations, and the like.

As shown in FIG. 4, the PPU 400 includes an Input/Output (I/O) unit 405, a front end unit 415, a scheduler unit 420, a work distribution unit 425, a hub 430, a crossbar (Xbar) 470, one or more general processing clusters (GPCs) 450, and one or more memory partition units 480. The PPU 400 may be connected to a host processor or other PPUs 400 via one or more high-speed NVLink 410 interconnect. The PPU 400 may be connected to a host processor or other peripheral devices via an interconnect 402. The PPU 400 may also be connected to a local memory 404 comprising a number of memory devices. In an embodiment, the local memory may comprise a number of dynamic random access memory (DRAM) devices. The DRAM devices may be configured as a high-bandwidth memory (HBM) subsystem, with multiple DRAM dies stacked within each device.

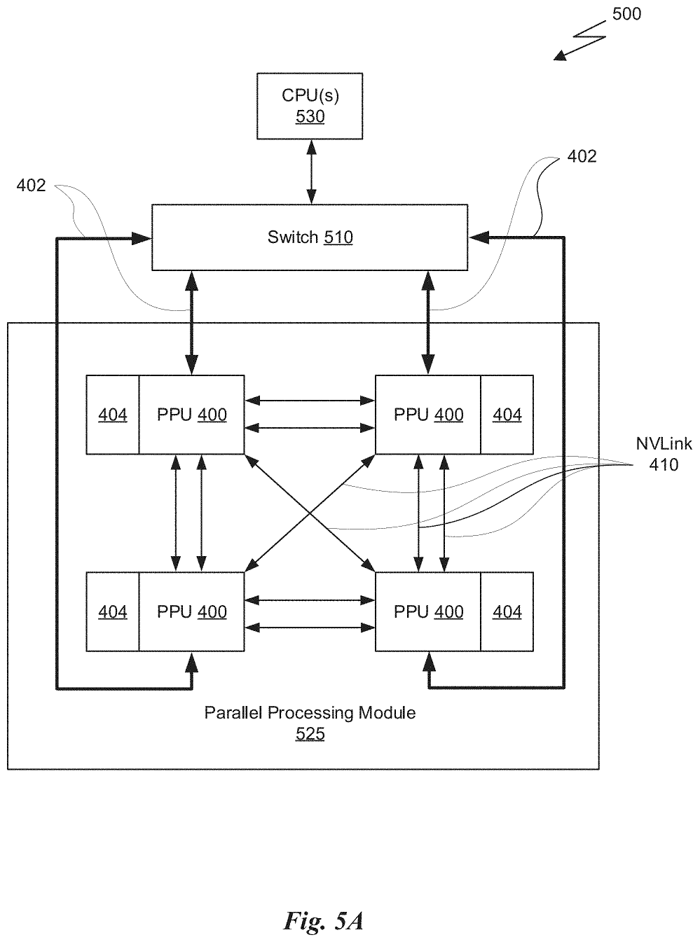

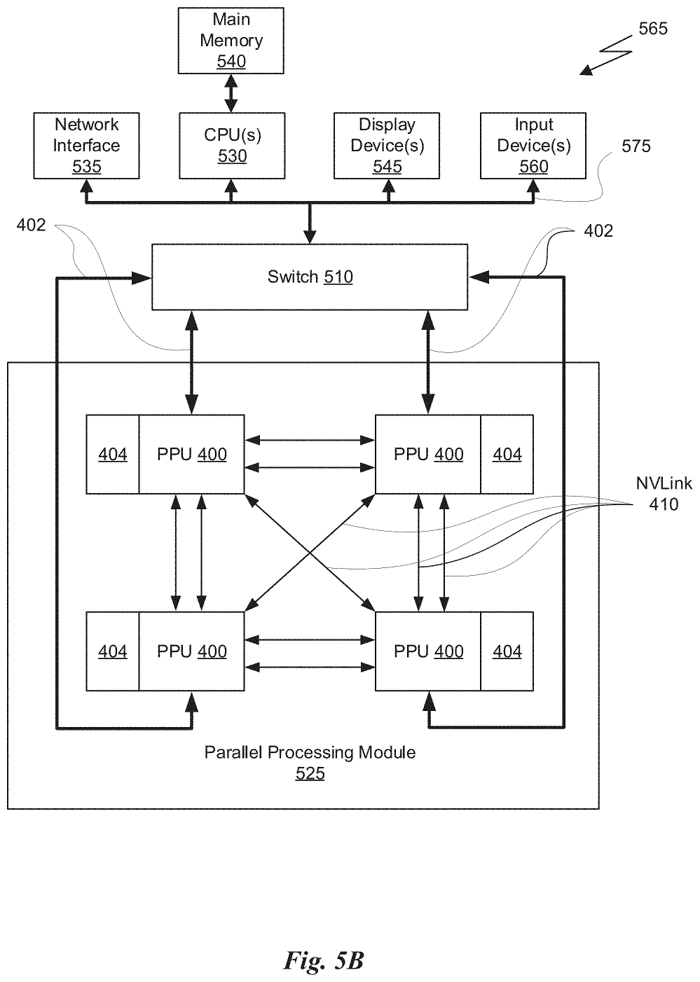

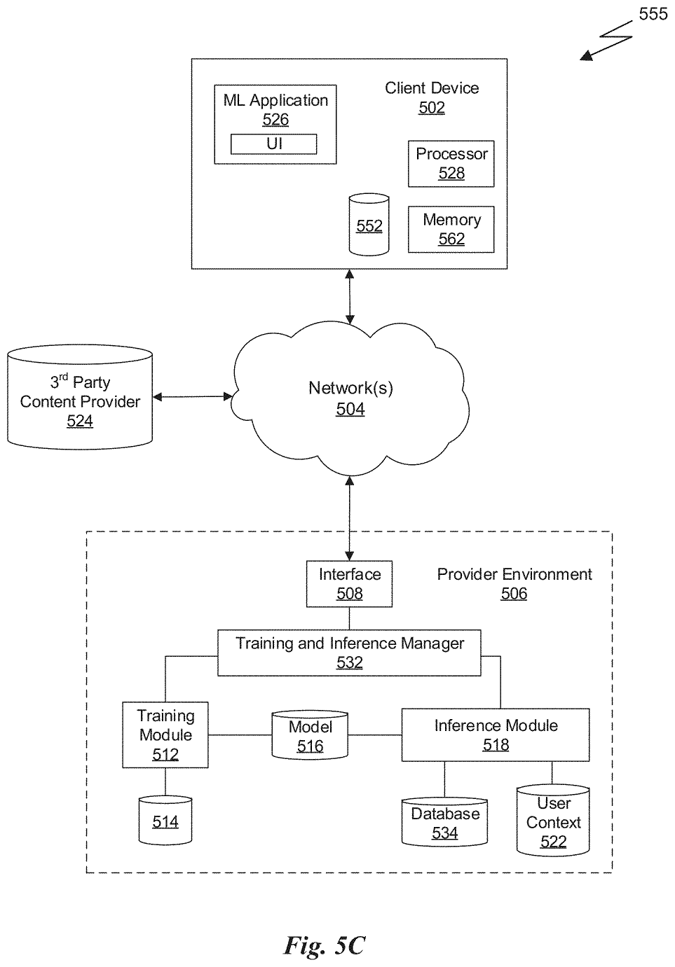

The NVLink 410 interconnect enables systems to scale and include one or more PPUs 400 combined with one or more CPUs, supports cache coherence between the PPUs 400 and CPUs, and CPU mastering. Data and/or commands may be transmitted by the NVLink 410 through the hub 430 to/from other units of the PPU 400 such as one or more copy engines, a video encoder, a video decoder, a power management unit, etc. (not explicitly shown). The NVLink 410 is described in more detail in conjunction with FIG. 5B.

The I/O unit 405 is configured to transmit and receive communications (e.g., commands, data, etc.) from a host processor (not shown) over the interconnect 402. The I/O unit 405 may communicate with the host processor directly via the interconnect 402 or through one or more intermediate devices such as a memory bridge. In an embodiment, the I/O unit 405 may communicate with one or more other processors, such as one or more the PPUs 400 via the interconnect 402. In an embodiment, the I/O unit 405 implements a Peripheral Component Interconnect Express (PCIe) interface for communications over a PCIe bus and the interconnect 402 is a PCIe bus. In alternative embodiments, the I/O unit 405 may implement other types of well-known interfaces for communicating with external devices.

The I/O unit 405 decodes packets received via the interconnect 402. In an embodiment, the packets represent commands configured to cause the PPU 400 to perform various operations. The I/O unit 405 transmits the decoded commands to various other units of the PPU 400 as the commands may specify. For example, some commands may be transmitted to the front end unit 415. Other commands may be transmitted to the hub 430 or other units of the PPU 400 such as one or more copy engines, a video encoder, a video decoder, a power management unit, etc. (not explicitly shown). In other words, the I/O unit 405 is configured to route communications between and among the various logical units of the PPU 400.

In an embodiment, a program executed by the host processor encodes a command stream in a buffer that provides workloads to the PPU 400 for processing. A workload may comprise several instructions and data to be processed by those instructions. The buffer is a region in a memory that is accessible (e.g., read/write) by both the host processor and the PPU 400. For example, the I/O unit 405 may be configured to access the buffer in a system memory connected to the interconnect 402 via memory requests transmitted over the interconnect 402. In an embodiment, the host processor writes the command stream to the buffer and then transmits a pointer to the start of the command stream to the PPU 400. The front end unit 415 receives pointers to one or more command streams. The front end unit 415 manages the one or more streams, reading commands from the streams and forwarding commands to the various units of the PPU 400.

The front end unit 415 is coupled to a scheduler unit 420 that configures the various GPCs 450 to process tasks defined by the one or more streams. The scheduler unit 420 is configured to track state information related to the various tasks managed by the scheduler unit 420. The state may indicate which GPC 450 a task is assigned to, whether the task is active or inactive, a priority level associated with the task, and so forth. The scheduler unit 420 manages the execution of a plurality of tasks on the one or more GPCs 450.

The scheduler unit 420 is coupled to a work distribution unit 425 that is configured to dispatch tasks for execution on the GPCs 450. The work distribution unit 425 may track a number of scheduled tasks received from the scheduler unit 420. In an embodiment, the work distribution unit 425 manages a pending task pool and an active task pool for each of the GPCs 450. As a GPC 450 finishes the execution of a task, that task is evicted from the active task pool for the GPC 450 and one of the other tasks from the pending task pool is selected and scheduled for execution on the GPC 450. If an active task has been idle on the GPC 450, such as while waiting for a data dependency to be resolved, then the active task may be evicted from the GPC 450 and returned to the pending task pool while another task in the pending task pool is selected and scheduled for execution on the GPC 450.

In an embodiment, a host processor executes a driver kernel that implements an application programming interface (API) that enables one or more applications executing on the host processor to schedule operations for execution on the PPU 400. In an embodiment, multiple compute applications are simultaneously executed by the PPU 400 and the PPU 400 provides isolation, quality of service (QoS), and independent address spaces for the multiple compute applications. An application may generate instructions (e.g., API calls) that cause the driver kernel to generate one or more tasks for execution by the PPU 400. The driver kernel outputs tasks to one or more streams being processed by the PPU 400. Each task may comprise one or more groups of related threads, referred to herein as a warp. In an embodiment, a warp comprises 32 related threads that may be executed in parallel. Cooperating threads may refer to a plurality of threads including instructions to perform the task and that may exchange data through shared memory. The tasks may be allocated to one or more processing units within a GPC 450 and instructions are scheduled for execution by at least one warp.

The work distribution unit 425 communicates with the one or more GPCs 450 via XBar 470. The XBar 470 is an interconnect network that couples many of the units of the PPU 400 to other units of the PPU 400. For example, the XBar 470 may be configured to couple the work distribution unit 425 to a particular GPC 450. Although not shown explicitly, one or more other units of the PPU 400 may also be connected to the XBar 470 via the hub 430.

The tasks are managed by the scheduler unit 420 and dispatched to a GPC 450 by the work distribution unit 425. The GPC 450 is configured to process the task and generate results. The results may be consumed by other tasks within the GPC 450, routed to a different GPC 450 via the XBar 470, or stored in the memory 404. The results can be written to the memory 404 via the memory partition units 480, which implement a memory interface for reading and writing data to/from the memory 404. The results can be transmitted to another PPU 400 or CPU via the NVLink 410. In an embodiment, the PPU 400 includes a number U of memory partition units 480 that is equal to the number of separate and distinct memory devices of the memory 404 coupled to the PPU 400. Each GPC 450 may include a memory management unit to provide translation of virtual addresses into physical addresses, memory protection, and arbitration of memory requests. In an embodiment, the memory management unit provides one or more translation lookaside buffers (TLBs) for performing translation of virtual addresses into physical addresses in the memory 404.

In an embodiment, the memory partition unit 480 includes a Raster Operations (ROP) unit, a level two (L2) cache, and a memory interface that is coupled to the memory 404. The memory interface may implement 32, 64, 128, 1024-bit data buses, or the like, for high-speed data transfer. The PPU 400 may be connected to up to Y memory devices, such as high bandwidth memory stacks or graphics double-data-rate, version 5, synchronous dynamic random access memory, or other types of persistent storage. In an embodiment, the memory interface implements an HBM2 memory interface and Y equals half U. In an embodiment, the HBM2 memory stacks are located on the same physical package as the PPU 400, providing substantial power and area savings compared with conventional GDDR5 SDRAM systems. In an embodiment, each HBM2 stack includes four memory dies and Y equals 4, with each HBM2 stack including two 128-bit channels per die for a total of 8 channels and a data bus width of 1024 bits.

In an embodiment, the memory 404 supports Single-Error Correcting Double-Error Detecting (SECDED) Error Correction Code (ECC) to protect data. ECC provides higher reliability for compute applications that are sensitive to data corruption. Reliability is especially important in large-scale cluster computing environments where PPUs 400 process very large datasets and/or run applications for extended periods.

In an embodiment, the PPU 400 implements a multi-level memory hierarchy. In an embodiment, the memory partition unit 480 supports a unified memory to provide a single unified virtual address space for CPU and PPU 400 memory, enabling data sharing between virtual memory systems. In an embodiment the frequency of accesses by a PPU 400 to memory located on other processors is traced to ensure that memory pages are moved to the physical memory of the PPU 400 that is accessing the pages more frequently. In an embodiment, the NVLink 410 supports address translation services allowing the PPU 400 to directly access a CPU's page tables and providing full access to CPU memory by the PPU 400.

In an embodiment, copy engines transfer data between multiple PPUs 400 or between PPUs 400 and CPUs. The copy engines can generate page faults for addresses that are not mapped into the page tables. The memory partition unit 480 can then service the page faults, mapping the addresses into the page table, after which the copy engine can perform the transfer. In a conventional system, memory is pinned (e.g., non-pageable) for multiple copy engine operations between multiple processors, substantially reducing the available memory. With hardware page faulting, addresses can be passed to the copy engines without worrying if the memory pages are resident, and the copy process is transparent.

Data from the memory 404 or other system memory may be fetched by the memory partition unit 480 and stored in the L2 cache 460, which is located on-chip and is shared between the various GPCs 450. As shown, each memory partition unit 480 includes a portion of the L2 cache associated with a corresponding memory 404. Lower level caches may then be implemented in various units within the GPCs 450. For example, each of the processing units within a GPC 450 may implement a level one (L1) cache. The L1 cache is private memory that is dedicated to a particular processing unit. The L2 cache 460 is coupled to the memory interface 470 and the XBar 470 and data from the L2 cache may be fetched and stored in each of the L1 caches for processing.

In an embodiment, the processing units within each GPC 450 implement a SIMD (Single-Instruction, Multiple-Data) architecture where each thread in a group of threads (e.g., a warp) is configured to process a different set of data based on the same set of instructions. All threads in the group of threads execute the same instructions. In another embodiment, the processing unit implements a SIMT (Single-Instruction, Multiple Thread) architecture where each thread in a group of threads is configured to process a different set of data based on the same set of instructions, but where individual threads in the group of threads are allowed to diverge during execution. In an embodiment, a program counter, call stack, and execution state is maintained for each warp, enabling concurrency between warps and serial execution within warps when threads within the warp diverge. In another embodiment, a program counter, call stack, and execution state is maintained for each individual thread, enabling equal concurrency between all threads, within and between warps. When execution state is maintained for each individual thread, threads executing the same instructions may be converged and executed in parallel for maximum efficiency.

Cooperative Groups is a programming model for organizing groups of communicating threads that allows developers to express the granularity at which threads are communicating, enabling the expression of richer, more efficient parallel decompositions. Cooperative launch APIs support synchronization amongst thread blocks for the execution of parallel algorithms. Conventional programming models provide a single, simple construct for synchronizing cooperating threads: a barrier across all threads of a thread block (e.g., the syncthreads( ) function). However, programmers would often like to define groups of threads at smaller than thread block granularities and synchronize within the defined groups to enable greater performance, design flexibility, and software reuse in the form of collective group-wide function interfaces.

Cooperative Groups enables programmers to define groups of threads explicitly at sub-block (e.g., as small as a single thread) and multi-block granularities, and to perform collective operations such as synchronization on the threads in a cooperative group. The programming model supports clean composition across software boundaries, so that libraries and utility functions can synchronize safely within their local context without having to make assumptions about convergence. Cooperative Groups primitives enable new patterns of cooperative parallelism, including producer-consumer parallelism, opportunistic parallelism, and global synchronization across an entire grid of thread blocks.

Each processing unit includes a large number (e.g., 128, etc.) of distinct processing cores (e.g., functional units) that may be fully-pipelined, single-precision, double-precision, and/or mixed precision and include a floating point arithmetic logic unit and an integer arithmetic logic unit. In an embodiment, the floating point arithmetic logic units implement the IEEE 754-2008 standard for floating point arithmetic. In an embodiment, the cores include 64 single-precision (32-bit) floating point cores, 64 integer cores, 32 double-precision (64-bit) floating point cores, and 8 tensor cores.

Tensor cores configured to perform matrix operations. In particular, the tensor cores are configured to perform deep learning matrix arithmetic, such as GEMM (matrix-matrix multiplication) for convolution operations during neural network training and inferencing. In an embodiment, each tensor core operates on a 4×4 matrix and performs a matrix multiply and accumulate operation D=A×B+C, where A, B, C, and D are 4×4 matrices.

In an embodiment, the matrix multiply inputs A and B may be integer, fixed-point, or floating point matrices, while the accumulation matrices C and D may be integer, fixed-point, or floating point matrices of equal or higher bitwidths. In an embodiment, tensor cores operate on one, four, or eight bit integer input data with 32-bit integer accumulation. The 8-bit integer matrix multiply requires 1024 operations and results in a full precision product that is then accumulated using 32-bit integer addition with the other intermediate products for a 8×8×16 matrix multiply. In an embodiment, tensor Cores operate on 16-bit floating point input data with 32-bit floating point accumulation. The 16-bit floating point multiply requires 64 operations and results in a full precision product that is then accumulated using 32-bit floating point addition with the other intermediate products for a 4×4×4 matrix multiply. In practice, Tensor Cores are used to perform much larger two-dimensional or higher dimensional matrix operations, built up from these smaller elements. An API, such as CUDA 9 C++ API, exposes specialized matrix load, matrix multiply and accumulate, and matrix store operations to efficiently use Tensor Cores from a CUDA-C++ program. At the CUDA level, the warp-level interface assumes 16×16 size matrices spanning all 32 threads of the warp.

Each processing unit may also comprise M special function units (SFUs) that perform special functions (e.g., attribute evaluation, reciprocal square root, and the like). In an embodiment, the SFUs may include a tree traversal unit configured to traverse a hierarchical tree data structure. In an embodiment, the SFUs may include texture unit configured to perform texture map filtering operations. In an embodiment, the texture units are configured to load texture maps (e.g., a 2D array of texels) from the memory 404 and sample the texture maps to produce sampled texture values for use in shader programs executed by the processing unit. In an embodiment, the texture maps are stored in shared memory that may comprise or include an L1 cache. The texture units implement texture operations such as filtering operations using mip-maps (e.g., texture maps of varying levels of detail). In an embodiment, each processing unit includes two texture units.

Each processing unit also comprises N load store units (LSUs) that implement load and store operations between the shared memory and the register file. Each processing unit includes an interconnect network that connects each of the cores to the register file and the LSU to the register file, shared memory. In an embodiment, the interconnect network is a crossbar that can be configured to connect any of the cores to any of the registers in the register file and connect the LSUs to the register file and memory locations in shared memory.

The shared memory is an array of on-chip memory that allows for data storage and communication between the processing units and between threads within a processing unit. In an embodiment, the shared memory comprises 128 KB of storage capacity and is in the path from each of the processing units to the memory partition unit 480. The shared memory can be used to cache reads and writes. One or more of the shared memory, L1 cache, L2 cache, and memory 404 are backing stores.

Combining data cache and shared memory functionality into a single memory block provides the best overall performance for both types of memory accesses. The capacity is usable as a cache by programs that do not use shared memory. For example, if shared memory is configured to use half of the capacity, texture and load/store operations can use the remaining capacity. Integration within the shared memory enables the shared memory to function as a high-throughput conduit for streaming data while simultaneously providing high-bandwidth and low-latency access to frequently reused data.

When configured for general purpose parallel computation, a simpler configuration can be used compared with graphics processing. Specifically, fixed function graphics processing units, are bypassed, creating a much simpler programming model. In the general purpose parallel computation configuration, the work distribution unit 425 assigns and distributes blocks of threads directly to the processing units within the GPCs 450. Threads execute the same program, using a unique thread ID in the calculation to ensure each thread generates unique results, using the processing unit(s) to execute the program and perform calculations, shared memory to communicate between threads, and the LSU to read and write global memory through the shared memory and the memory partition unit 480. When configured for general purpose parallel computation, the processing units can also write commands that the scheduler unit 420 can use to launch new work on the processing units.

The PPUs 400 may each include, and/or be configured to perform functions of, one or more processing cores and/or components thereof, such as Tensor Cores (TCs), Tensor Processing Units (TPUs), Pixel Visual Cores (PVCs), Ray Tracing (RT) Cores, Vision Processing Units (VPUs), Graphics Processing Clusters (GPCs), Texture Processing Clusters (TPCs), Streaming Multiprocessors (SMs), Tree Traversal Units (TTUs), Artificial Intelligence Accelerators (AIAs), Deep Learning Accelerators (DLAs), Arithmetic-Logic Units (ALUs), Application-Specific Integrated Circuits (ASICs), Floating Point Units (FPUs), input/output (I/O) elements, peripheral component interconnect (PCI) or peripheral component interconnect express (PCIe) elements, and/or the like.

The PPU 400 may be included in a desktop computer, a laptop computer, a tablet computer, servers, supercomputers, a smart-phone (e.g., a wireless, hand-held device), personal digital assistant (PDA), a digital camera, a vehicle, a head mounted display, a hand-held electronic device, and the like. In an embodiment, the PPU 400 is embodied on a single semiconductor substrate. In another embodiment, the PPU 400 is included in a system-on-a-chip (SoC) along with one or more other devices such as additional PPUs 400, the memory 404, a reduced instruction set computer (RISC) CPU, a memory management unit (MMU), a digital-to-analog converter (DAC), and the like.

In an embodiment, the PPU 400 may be included on a graphics card that includes one or more memory devices. The graphics card may be configured to interface with a PCIe slot on a motherboard of a desktop computer. In yet another embodiment, the PPU 400 may be an integrated graphics processing unit (iGPU) or parallel processor included in the chipset of the motherboard. In yet another embodiment, the PPU 400 may be realized in reconfigurable hardware. In yet another embodiment, parts of the PPU 400 may be realized in reconfigurable hardware.

Exemplary Computing System

Systems with multiple GPUs and CPUs are used in a variety of industries as developers expose and leverage more parallelism in applications such as artificial intelligence computing. High-performance GPU-accelerated systems with tens to many thousands of compute nodes are deployed in data centers, research facilities, and supercomputers to solve ever larger problems. As the number of processing devices within the high-performance systems increases, the communication and data transfer mechanisms need to scale to support the increased bandwidth.

FIG. 5A is a conceptual diagram of a processing system 500 implemented using the PPU 400 of FIG. 4, in accordance with an embodiment. The exemplary system 500 may be configured to implement the method 100 shown in FIG. 1. The processing system 500 includes a CPU 530, switch 510, and multiple PPUs 400, and respective memories 404.

The NVLink 410 provides high-speed communication links between each of the PPUs 400. Although a particular number of NVLink 410 and interconnect 402 connections are illustrated in FIG. 5B, the number of connections to each PPU 400 and the CPU 530 may vary. The switch 510 interfaces between the interconnect 402 and the CPU 530. The PPUs 400, memories 404, and NVLinks 410 may be situated on a single semiconductor platform to form a parallel processing module 525. In an embodiment, the switch 510 supports two or more protocols to interface between various different connections and/or links.

In another embodiment (not shown), the NVLink 410 provides one or more high-speed communication links between each of the PPUs 400 and the CPU 530 and the switch 510 interfaces between the interconnect 402 and each of the PPUs 400. The PPUs 400, memories 404, and interconnect 402 may be situated on a single semiconductor platform to form a parallel processing module 525. In yet another embodiment (not shown), the interconnect 402 provides one or more communication links between each of the PPUs 400 and the CPU 530 and the switch 510 interfaces between each of the PPUs 400 using the NVLink 410 to provide one or more high-speed communication links between the PPUs 400. In another embodiment (not shown), the NVLink 410 provides one or more high-speed communication links between the PPUs 400 and the CPU 530 through the switch 510. In yet another embodiment (not shown), the interconnect 402 provides one or more communication links between each of the PPUs 400 directly. One or more of the NVLink 410 high-speed communication links may be implemented as a physical NVLink interconnect or either an on-chip or on-die interconnect using the same protocol as the NVLink 410.

In the context of the present description, a single semiconductor platform may refer to a sole unitary semiconductor-based integrated circuit fabricated on a die or chip. It should be noted that the term single semiconductor platform may also refer to multi-chip modules with increased connectivity which simulate on-chip operation and make substantial improvements over utilizing a conventional bus implementation. Of course, the various circuits or devices may also be situated separately or in various combinations of semiconductor platforms per the desires of the user. Alternately, the parallel processing module 525 may be implemented as a circuit board substrate and each of the PPUs 400 and/or memories 404 may be packaged devices. In an embodiment, the CPU 530, switch 510, and the parallel processing module 525 are situated on a single semiconductor platform.

In an embodiment, the signaling rate of each NVLink 410 is 20 to 25 Gigabits/second and each PPU 400 includes six NVLink 410 interfaces (as shown in FIG. 5A, five NVLink 410 interfaces are included for each PPU 400). Each NVLink 410 provides a data transfer rate of 25 Gigabytes/second in each direction, with six links providing 400 Gigabytes/second. The NVLinks 410 can be used exclusively for PPU-to-PPU communication as shown in FIG. 5A, or some combination of PPU-to-PPU and PPU-to-CPU, when the CPU 530 also includes one or more NVLink 410 interfaces.

In an embodiment, the NVLink 410 allows direct load/store/atomic access from the CPU 530 to each PPU's 400 memory 404. In an embodiment, the NVLink 410 supports coherency operations, allowing data read from the memories 404 to be stored in the cache hierarchy of the CPU 530, reducing cache access latency for the CPU 530. In an embodiment, the NVLink 410 includes support for Address Translation Services (ATS), allowing the PPU 400 to directly access page tables within the CPU 530. One or more of the NVLinks 410 may also be configured to operate in a low-power mode.

FIG. 5B illustrates an exemplary system 565 in which the various architecture and/or functionality of the various previous embodiments may be implemented. The exemplary system 565 may be configured to implement the method 100 shown in FIG. 1.

As shown, a system 565 is provided including at least one central processing unit 530 that is connected to a communication bus 575. The communication bus 575 may directly or indirectly couple one or more of the following devices: main memory 540, network interface 535, CPU(s) 530, display device(s) 545, input device(s) 560, switch 510, and parallel processing system 525. The communication bus 575 may be implemented using any suitable protocol and may represent one or more links or busses, such as an address bus, a data bus, a control bus, or a combination thereof. The communication bus 575 may include one or more bus or link types, such as an industry standard architecture (ISA) bus, an extended industry standard architecture (EISA) bus, a video electronics standards association (VESA) bus, a peripheral component interconnect (PCI) bus, a peripheral component interconnect express (PCIe) bus, HyperTransport, and/or another type of bus or link. In some embodiments, there are direct connections between components. As an example, the CPU(s) 530 may be directly connected to the main memory 540. Further, the CPU(s) 530 may be directly connected to the parallel processing system 525. Where there is direct, or point-to-point connection between components, the communication bus 575 may include a PCIe link to carry out the connection. In these examples, a PCI bus need not be included in the system 565.

Although the various blocks of FIG. 5B are shown as connected via the communication bus 575 with lines, this is not intended to be limiting and is for clarity only. For example, in some embodiments, a presentation component, such as display device(s) 545, may be considered an I/O component, such as input device(s) 560 (e.g., if the display is a touch screen). As another example, the CPU(s) 530 and/or parallel processing system 525 may include memory (e.g., the main memory 540 may be representative of a storage device in addition to the parallel processing system 525, the CPUs 530, and/or other components). In other words, the computing device of FIG. 5B is merely illustrative. Distinction is not made between such categories as “workstation,” “server,” “laptop,” “desktop,” “tablet,” “client device,” “mobile device,” “hand-held device,” “game console,” “electronic control unit (ECU),” “virtual reality system,” and/or other device or system types, as all are contemplated within the scope of the computing device of FIG. 5B.

The system 565 also includes a main memory 540. Control logic (software) and data are stored in the main memory 540 which may take the form of a variety of computer-readable media. The computer-readable media may be any available media that may be accessed by the system 565. The computer-readable media may include both volatile and nonvolatile media, and removable and non-removable media. By way of example, and not limitation, the computer-readable media may comprise computer-storage media and communication media.

The computer-storage media may include both volatile and nonvolatile media and/or removable and non-removable media implemented in any method or technology for storage of information such as computer-readable instructions, data structures, program modules, and/or other data types. For example, the main memory 540 may store computer-readable instructions (e.g., that represent a program(s) and/or a program element(s), such as an operating system. Computer-storage media may include, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which may be used to store the desired information and which may be accessed by system 565. As used herein, computer storage media does not comprise signals per se.

The computer storage media may embody computer-readable instructions, data structures, program modules, and/or other data types in a modulated data signal such as a carrier wave or other transport mechanism and includes any information delivery media. The term “modulated data signal” may refer to a signal that has one or more of its characteristics set or changed in such a manner as to encode information in the signal. By way of example, and not limitation, the computer storage media may include wired media such as a wired network or direct-wired connection, and wireless media such as acoustic, RF, infrared and other wireless media. Combinations of any of the above should also be included within the scope of computer-readable media.

Computer programs, when executed, enable the system 565 to perform various functions. The CPU(s) 530 may be configured to execute at least some of the computer-readable instructions to control one or more components of the system 565 to perform one or more of the methods and/or processes described herein. The CPU(s) 530 may each include one or more cores (e.g., one, two, four, eight, twenty-eight, seventy-two, etc.) that are capable of handling a multitude of software threads simultaneously. The CPU(s) 530 may include any type of processor, and may include different types of processors depending on the type of system 565 implemented (e.g., processors with fewer cores for mobile devices and processors with more cores for servers). For example, depending on the type of system 565, the processor may be an Advanced RISC Machines (ARM) processor implemented using Reduced Instruction Set Computing (RISC) or an x86 processor implemented using Complex Instruction Set Computing (CISC). The system 565 may include one or more CPUs 530 in addition to one or more microprocessors or supplementary co-processors, such as math co-processors.

In addition to or alternatively from the CPU(s) 530, the parallel processing module 525 may be configured to execute at least some of the computer-readable instructions to control one or more components of the system 565 to perform one or more of the methods and/or processes described herein. The parallel processing module 525 may be used by the system 565 to render graphics (e.g., 3D graphics) or perform general purpose computations. For example, the parallel processing module 525 may be used for General-Purpose computing on GPUs (GPGPU). In embodiments, the CPU(s) 530 and/or the parallel processing module 525 may discretely or jointly perform any combination of the methods, processes and/or portions thereof.

The system 565 also includes input device(s) 560, the parallel processing system 525, and display device(s) 545. The display device(s) 545 may include a display (e.g., a monitor, a touch screen, a television screen, a heads-up-display (HUD), other display types, or a combination thereof), speakers, and/or other presentation components. The display device(s) 545 may receive data from other components (e.g., the parallel processing system 525, the CPU(s) 530, etc.), and output the data (e.g., as an image, video, sound, etc.).

The network interface 535 may enable the system 565 to be logically coupled to other devices including the input devices 560, the display device(s) 545, and/or other components, some of which may be built in to (e.g., integrated in) the system 565. Illustrative input devices 560 include a microphone, mouse, keyboard, joystick, game pad, game controller, satellite dish, scanner, printer, wireless device, etc. The input devices 560 may provide a natural user interface (NUI) that processes air gestures, voice, or other physiological inputs generated by a user. In some instances, inputs may be transmitted to an appropriate network element for further processing. An NUI may implement any combination of speech recognition, stylus recognition, facial recognition, biometric recognition, gesture recognition both on screen and adjacent to the screen, air gestures, head and eye tracking, and touch recognition (as described in more detail below) associated with a display of the system 565. The system 565 may be include depth cameras, such as stereoscopic camera systems, infrared camera systems, RGB camera systems, touchscreen technology, and combinations of these, for gesture detection and recognition. Additionally, the system 565 may include accelerometers or gyroscopes (e.g., as part of an inertia measurement unit (IMU)) that enable detection of motion. In some examples, the output of the accelerometers or gyroscopes may be used by the system 565 to render immersive augmented reality or virtual reality.

Further, the system 565 may be coupled to a network (e.g., a telecommunications network, local area network (LAN), wireless network, wide area network (WAN) such as the Internet, peer-to-peer network, cable network, or the like) through a network interface 535 for communication purposes. The system 565 may be included within a distributed network and/or cloud computing environment.

The network interface 535 may include one or more receivers, transmitters, and/or transceivers that enable the system 565 to communicate with other computing devices via an electronic communication network, included wired and/or wireless communications. The network interface 535 may include components and functionality to enable communication over any of a number of different networks, such as wireless networks (e.g., Wi-Fi, Z-Wave, Bluetooth, Bluetooth LE, ZigBee, etc.), wired networks (e.g., communicating over Ethernet or InfiniBand), low-power wide-area networks (e.g., LoRaWAN, SigFox, etc.), and/or the Internet.

The system 565 may also include a secondary storage (not shown). The secondary storage includes, for example, a hard disk drive and/or a removable storage drive, representing a floppy disk drive, a magnetic tape drive, a compact disk drive, digital versatile disk (DVD) drive, recording device, universal serial bus (USB) flash memory. The removable storage drive reads from and/or writes to a removable storage unit in a well-known manner. The system 565 may also include a hard-wired power supply, a battery power supply, or a combination thereof (not shown). The power supply may provide power to the system 565 to enable the components of the system 565 to operate.

Each of the foregoing modules and/or devices may even be situated on a single semiconductor platform to form the system 565. Alternately, the various modules may also be situated separately or in various combinations of semiconductor platforms per the desires of the user. While various embodiments have been described above, it should be understood that they have been presented by way of example only, and not limitation. Thus, the breadth and scope of a preferred embodiment should not be limited by any of the above-described exemplary embodiments, but should be defined only in accordance with the following claims and their equivalents.

Example Network Environments

Network environments suitable for use in implementing embodiments of the disclosure may include one or more client devices, servers, network attached storage (NAS), other backend devices, and/or other device types. The client devices, servers, and/or other device types (e.g., each device) may be implemented on one or more instances of the processing system 500 of FIG. 5A and/or exemplary system 565 of FIG. 5B—e.g., each device may include similar components, features, and/or functionality of the processing system 500 and/or exemplary system 565.

Components of a network environment may communicate with each other via a network(s), which may be wired, wireless, or both. The network may include multiple networks, or a network of networks. By way of example, the network may include one or more Wide Area Networks (WANs), one or more Local Area Networks (LANs), one or more public networks such as the Internet and/or a public switched telephone network (PSTN), and/or one or more private networks. Where the network includes a wireless telecommunications network, components such as a base station, a communications tower, or even access points (as well as other components) may provide wireless connectivity.

Compatible network environments may include one or more peer-to-peer network environments—in which case a server may not be included in a network environment—and one or more client-server network environments—in which case one or more servers may be included in a network environment. In peer-to-peer network environments, functionality described herein with respect to a server(s) may be implemented on any number of client devices.

In at least one embodiment, a network environment may include one or more cloud-based network environments, a distributed computing environment, a combination thereof, etc. A cloud-based network environment may include a framework layer, a job scheduler, a resource manager, and a distributed file system implemented on one or more of servers, which may include one or more core network servers and/or edge servers. A framework layer may include a framework to support software of a software layer and/or one or more application(s) of an application layer. The software or application(s) may respectively include web-based service software or applications. In embodiments, one or more of the client devices may use the web-based service software or applications (e.g., by accessing the service software and/or applications via one or more application programming interfaces (APIs)). The framework layer may be, but is not limited to, a type of free and open-source software web application framework such as that may use a distributed file system for large-scale data processing (e.g., “big data”).

A cloud-based network environment may provide cloud computing and/or cloud storage that carries out any combination of computing and/or data storage functions described herein (or one or more portions thereof). Any of these various functions may be distributed over multiple locations from central or core servers (e.g., of one or more data centers that may be distributed across a state, a region, a country, the globe, etc.). If a connection to a user (e.g., a client device) is relatively close to an edge server(s), a core server(s) may designate at least a portion of the functionality to the edge server(s). A cloud-based network environment may be private (e.g., limited to a single organization), may be public (e.g., available to many organizations), and/or a combination thereof (e.g., a hybrid cloud environment).

The client device(s) may include at least some of the components, features, and functionality of the example processing system 500 of FIG. 5A and/or exemplary system 565 of FIG. 5B. By way of example and not limitation, a client device may be embodied as a Personal Computer (PC), a laptop computer, a mobile device, a smartphone, a tablet computer, a smart watch, a wearable computer, a Personal Digital Assistant (PDA), an MP3 player, a virtual reality headset, a Global Positioning System (GPS) or device, a video player, a video camera, a surveillance device or system, a vehicle, a boat, a flying vessel, a virtual machine, a drone, a robot, a handheld communications device, a hospital device, a gaming device or system, an entertainment system, a vehicle computer system, an embedded system controller, a remote control, an appliance, a consumer electronic device, a workstation, an edge device, any combination of these delineated devices, or any other suitable device.

Machine Learning

Deep neural networks (DNNs) developed on processors, such as the PPU 400 have been used for diverse use cases, from self-driving cars to faster drug development, from automatic image captioning in online image databases to smart real-time language translation in video chat applications. Deep learning is a technique that models the neural learning process of the human brain, continually learning, continually getting smarter, and delivering more accurate results more quickly over time. A child is initially taught by an adult to correctly identify and classify various shapes, eventually being able to identify shapes without any coaching. Similarly, a deep learning or neural learning system needs to be trained in object recognition and classification for it get smarter and more efficient at identifying basic objects, occluded objects, etc., while also assigning context to objects.

At the simplest level, neurons in the human brain look at various inputs that are received, importance levels are assigned to each of these inputs, and output is passed on to other neurons to act upon. An artificial neuron or perceptron is the most basic model of a neural network. In one example, a perceptron may receive one or more inputs that represent various features of an object that the perceptron is being trained to recognize and classify, and each of these features is assigned a certain weight based on the importance of that feature in defining the shape of an object.

A deep neural network (DNN) model includes multiple layers of many connected nodes (e.g., perceptrons, Boltzmann machines, radial basis functions, convolutional layers, etc.) that can be trained with enormous amounts of input data to quickly solve complex problems with high accuracy. In one example, a first layer of the DNN model breaks down an input image of an automobile into various sections and looks for basic patterns such as lines and angles. The second layer assembles the lines to look for higher level patterns such as wheels, windshields, and mirrors. The next layer identifies the type of vehicle, and the final few layers generate a label for the input image, identifying the model of a specific automobile brand.

Once the DNN is trained, the DNN can be deployed and used to identify and classify objects or patterns in a process known as inference. Examples of inference (the process through which a DNN extracts useful information from a given input) include identifying handwritten numbers on checks deposited into ATM machines, identifying images of friends in photos, delivering movie recommendations to over fifty million users, identifying and classifying different types of automobiles, pedestrians, and road hazards in driverless cars, or translating human speech in real-time.

During training, data flows through the DNN in a forward propagation phase until a prediction is produced that indicates a label corresponding to the input. If the neural network does not correctly label the input, then errors between the correct label and the predicted label are analyzed, and the weights are adjusted for each feature during a backward propagation phase until the DNN correctly labels the input and other inputs in a training dataset. Training complex neural networks requires massive amounts of parallel computing performance, including floating-point multiplications and additions that are supported by the PPU 400. Inferencing is less compute-intensive than training, being a latency-sensitive process where a trained neural network is applied to new inputs it has not seen before to classify images, detect emotions, identify recommendations, recognize and translate speech, and generally infer new information.

Neural networks rely heavily on matrix math operations, and complex multi-layered networks require tremendous amounts of floating-point performance and bandwidth for both efficiency and speed. With thousands of processing cores, optimized for matrix math operations, and delivering tens to hundreds of TFLOPS of performance, the PPU 400 is a computing platform capable of delivering performance required for deep neural network-based artificial intelligence and machine learning applications.

FIG. 5C illustrates components of an exemplary system 555 that can be used to train and utilize machine learning, in accordance with at least one embodiment. As will be discussed, various components can be provided by various combinations of computing devices and resources, or a single computing system, which may be under control of a single entity or multiple entities. Further, aspects may be triggered, initiated, or requested by different entities. In at least one embodiment training of a neural network might be instructed by a provider associated with provider environment 506, while in at least one embodiment training might be requested by a customer or other user having access to a provider environment through a client device 502 or other such resource. In at least one embodiment, training data (or data to be analyzed by a trained neural network) can be provided by a provider, a user, or a third party content provider 524. In at least one embodiment, client device 502 may be a vehicle or object that is to be navigated on behalf of a user, for example, which can submit requests and/or receive instructions that assist in navigation of a device.

In at least one embodiment, requests are able to be submitted across at least one network 504 to be received by a provider environment 506. In at least one embodiment, a client device may be any appropriate electronic and/or computing devices enabling a user to generate and send such requests, such as, but not limited to, desktop computers, notebook computers, computer servers, smartphones, tablet computers, gaming consoles (portable or otherwise), computer processors, computing logic, and set-top boxes. Network(s) 504 can include any appropriate network for transmitting a request or other such data, as may include Internet, an intranet, an Ethernet, a cellular network, a local area network (LAN), a wide area network (WAN), a personal area network (PAN), an ad hoc network of direct wireless connections among peers, and so on.

In at least one embodiment, requests can be received at an interface layer 508, which can forward data to a training and inference manager 532, in this example. The training and inference manager 532 can be a system or service including hardware and software for managing requests and service corresponding data or content, in at least one embodiment, the training and inference manager 532 can receive a request to train a neural network, and can provide data for a request to a training module 512. In at least one embodiment, training module 512 can select an appropriate model or neural network to be used, if not specified by the request, and can train a model using relevant training data. In at least one embodiment, training data can be a batch of data stored in a training data repository 514, received from client device 502, or obtained from a third party provider 524. In at least one embodiment, training module 512 can be responsible for training data. A neural network can be any appropriate network, such as a recurrent neural network (RNN) or convolutional neural network (CNN). Once a neural network is trained and successfully evaluated, a trained neural network can be stored in a model repository 516, for example, that may store different models or networks for users, applications, or services, etc. In at least one embodiment, there may be multiple models for a single application or entity, as may be utilized based on a number of different factors.

In at least one embodiment, at a subsequent point in time, a request may be received from client device 502 (or another such device) for content (e.g., path determinations) or data that is at least partially determined or impacted by a trained neural network. This request can include, for example, input data to be processed using a neural network to obtain one or more inferences or other output values, classifications, or predictions, or for at least one embodiment, input data can be received by interface layer 508 and directed to inference module 518, although a different system or service can be used as well. In at least one embodiment, inference module 518 can obtain an appropriate trained network, such as a trained deep neural network (DNN) as discussed herein, from model repository 516 if not already stored locally to inference module 518. Inference module 518 can provide data as input to a trained network, which can then generate one or more inferences as output. This may include, for example, a classification of an instance of input data. In at least one embodiment, inferences can then be transmitted to client device 502 for display or other communication to a user. In at least one embodiment, context data for a user may also be stored to a user context data repository 522, which may include data about a user which may be useful as input to a network in generating inferences, or determining data to return to a user after obtaining instances. In at least one embodiment, relevant data, which may include at least some of input or inference data, may also be stored to a local database 534 for processing future requests. In at least one embodiment, a user can use account information or other information to access resources or functionality of a provider environment. In at least one embodiment, if permitted and available, user data may also be collected and used to further train models, in order to provide more accurate inferences for future requests. In at least one embodiment, requests may be received through a user interface to a machine learning application 526 executing on client device 502, and results displayed through a same interface. A client device can include resources such as a processor 528 and memory 562 for generating a request and processing results or a response, as well as at least one data storage element 552 for storing data for machine learning application 526.

In at least one embodiment a processor 528 (or a processor of training module 512 or inference module 518) will be a central processing unit (CPU). As mentioned, however, resources in such environments can utilize GPUs to process data for at least certain types of requests. With thousands of cores, GPUs, such as PPU 300 are designed to handle substantial parallel workloads and, therefore, have become popular in deep learning for training neural networks and generating predictions. While use of GPUs for offline builds has enabled faster training of larger and more complex models, generating predictions offline implies that either request-time input features cannot be used or predictions must be generated for all permutations of features and stored in a lookup table to serve real-time requests. If a deep learning framework supports a CPU-mode and a model is small and simple enough to perform a feed-forward on a CPU with a reasonable latency, then a service on a CPU instance could host a model. In this case, training can be done offline on a GPU and inference done in real-time on a CPU. If a CPU approach is not viable, then a service can run on a GPU instance. Because GPUs have different performance and cost characteristics than CPUs, however, running a service that offloads a runtime algorithm to a GPU can require it to be designed differently from a CPU based service.

In at least one embodiment, video data can be provided from client device 502 for enhancement in provider environment 506. In at least one embodiment, video data can be processed for enhancement on client device 502. In at least one embodiment, video data may be streamed from a third party content provider 524 and enhanced by third party content provider 524, provider environment 506, or client device 502. In at least one embodiment, video data can be provided from client device 502 for use as training data in provider environment 506.

In at least one embodiment, supervised and/or unsupervised training can be performed by the client device 502 and/or the provider environment 506. In at least one embodiment, a set of training data 514 (e.g., classified or labeled data) is provided as input to function as training data.

In at least one embodiment, training data can include instances of at least one type of object for which a neural network is to be trained, as well as information that identifies that type of object. In at least one embodiment, training data might include a set of images that each includes a representation of a type of object, where each image also includes, or is associated with, a label, metadata, classification, or other piece of information identifying a type of object represented in a respective image. Various other types of data may be used as training data as well, as may include text data, audio data, video data, and so on. In at least one embodiment, training data 514 is provided as training input to a training module 512. In at least one embodiment, training module 512 can be a system or service that includes hardware and software, such as one or more computing devices executing a training application, for training a neural network (or other model or algorithm, etc.). In at least one embodiment, training module 512 receives an instruction or request indicating a type of model to be used for training, in at least one embodiment, a model can be any appropriate statistical model, network, or algorithm useful for such purposes, as may include an artificial neural network, deep learning algorithm, learning classifier, Bayesian network, and so on. In at least one embodiment, training module 512 can select an initial model, or other untrained model, from an appropriate repository 516 and utilize training data 514 to train a model, thereby generating a trained model (e.g., trained deep neural network) that can be used to classify similar types of data, or generate other such inferences. In at least one embodiment where training data is not used, an appropriate initial model can still be selected for training on input data per training module 512.

In at least one embodiment, a model can be trained in a number of different ways, as may depend in part upon a type of model selected. In at least one embodiment, a machine learning algorithm can be provided with a set of training data, where a model is a model artifact created by a training process. In at least one embodiment, each instance of training data contains a correct answer (e.g., classification), which can be referred to as a target or target attribute. In at least one embodiment, a learning algorithm finds patterns in training data that map input data attributes to a target, an answer to be predicted, and a machine learning model is output that captures these patterns. In at least one embodiment, a machine learning model can then be used to obtain predictions on new data for which a target is not specified.

In at least one embodiment, training and inference manager 532 can select from a set of machine learning models including binary classification, multiclass classification, generative, and regression models. In at least one embodiment, a type of model to be used can depend at least in part upon a type of target to be predicted.

It is noted that the techniques described herein may be embodied in executable instructions stored in a computer readable medium for use by or in connection with a processor-based instruction execution machine, system, apparatus, or device. It will be appreciated by those skilled in the art that, for some embodiments, various types of computer-readable media can be included for storing data. As used herein, a “computer-readable medium” includes one or more of any suitable media for storing the executable instructions of a computer program such that the instruction execution machine, system, apparatus, or device may read (or fetch) the instructions from the computer-readable medium and execute the instructions for carrying out the described embodiments. Suitable storage formats include one or more of an electronic, magnetic, optical, and electromagnetic format. A non-exhaustive list of conventional exemplary computer-readable medium includes: a portable computer diskette; a random-access memory (RAM); a read-only memory (ROM); an erasable programmable read only memory (EPROM); a flash memory device; and optical storage devices, including a portable compact disc (CD), a portable digital video disc (DVD), and the like.

It should be understood that the arrangement of components illustrated in the attached Figures are for illustrative purposes and that other arrangements are possible. For example, one or more of the elements described herein may be realized, in whole or in part, as an electronic hardware component. Other elements may be implemented in software, hardware, or a combination of software and hardware. Moreover, some or all of these other elements may be combined, some may be omitted altogether, and additional components may be added while still achieving the functionality described herein. Thus, the subject matter described herein may be embodied in many different variations, and all such variations are contemplated to be within the scope of the claims.

To facilitate an understanding of the subject matter described herein, many aspects are described in terms of sequences of actions. It will be recognized by those skilled in the art that the various actions may be performed by specialized circuits or circuitry, by program instructions being executed by one or more processors, or by a combination of both. The description herein of any sequence of actions is not intended to imply that the specific order described for performing that sequence must be followed. All methods described herein may be performed in any suitable order unless otherwise indicated herein or otherwise clearly contradicted by context.

The use of the terms “a” and “an” and “the” and similar references in the context of describing the subject matter (particularly in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. Furthermore, the foregoing description is for the purpose of illustration only, and not for the purpose of limitation, as the scope of protection sought is defined by the claims as set forth hereinafter together with any equivalents thereof. The use of any and all examples, or exemplary language (e.g., “such as”) provided herein, is intended merely to better illustrate the subject matter and does not pose a limitation on the scope of the subject matter unless otherwise claimed. The use of the term “based on” and other like phrases indicating a condition for bringing about a result, both in the claims and in the written description, is not intended to foreclose any other conditions that bring about that result. No language in the specification should be construed as indicating any non-claimed element as essential to the practice of the invention as claimed.

1. A method for simulating a cutting operation, the method comprising:
receiving a mesh for an object;
modifying the mesh to add virtual nodes associated with a predefined cutting plane;
optimizing a set of parameters associated with a simulator based on ground-truth data; and
running a simulation via the simulator to generate outputs that include trajectories associated with a cutting instrument.

2. The method of claim 1, wherein the set of parameters includes a set of spring constants for a plurality of virtual springs, each virtual spring associated with two virtual nodes.

3. The method of claim 2, wherein, during the simulation, the set of spring constants are updated during a plurality of time steps based on contact forces calculated between the cutting instrument and the object.

4. The method of claim 2, wherein running the simulation comprises, for each time step in a plurality of time steps:
calculating external contact forces between the object and a surface due to gravity;
calculating elastic forces for tetrahedral elements of the mesh based on a strain energy density formula;
calculating contact forces between the object and the cutting instrument;
updating the set of spring constants based on the contact forces; and
calculating spring forces associated with the virtual springs.

5. The method of claim 4, wherein the contact forces include contact normal forces and friction forces derived based on a signed distance function (SDF) representation of the cutting instrument.

6. The method of claim 1, wherein the trajectories comprise an estimate of a force associated with the cutting instrument over a period of time.

7. The method of claim 6, wherein optimizing the set of parameters comprises applying stochastic gradient descent based on a set of ground-truth trajectories captured using sensors to measure real-world cutting operations.

8. The method of claim 1, wherein optimizing the set of parameters comprises applying an Adaptive Moment Estimation (Adam) optimizer to compute the set of parameters for the simulation.

9. The method of claim 1, wherein optimizing the set of parameters comprises applying a stochastic gradient Langevin dynamics (SGLD) algorithm to compute the set of parameters for the simulation.

10. The method of claim 1, wherein optimizing the set of parameters comprises applying a BayesSim algorithm to compute the set of parameters for the simulation.

11. The method of claim 1, further comprising generating control signals for a robot based on the outputs of the simulation.

12. The method of claim 1, wherein optimizing the set of parameters comprises optimizing a trajectory of a cutting instrument using gradient-based descent and a Modified Differential Method of Multipliers (MDMM).

13. A system for simulating a cutting operation, the system comprising:
a memory storing a mesh for an object and a set of parameters associated with a simulator; and
one or more processors coupled to the memory and configured to:
modify the mesh to add virtual nodes associated with a predefined cutting plane,
optimize the set of parameters based on ground-truth data, and
run a simulation via the simulator to generate outputs that include trajectories associated with a cutting instrument.

14. The system of claim 13, wherein running the simulation comprises, for each time step in a plurality of time steps:
calculating external contact forces between the object and a surface due to gravity;
calculating elastic forces for tetrahedral elements of the mesh based on a strain energy density formula;
calculating contact forces between the object and the cutting instrument;
updating the set of spring constants based on the contact forces; and
calculating spring forces associated with the virtual springs.

15. The system of claim 13, the system further comprising:
a robot, wherein the one or more processors are further configured to generate control signals for the robot based on the outputs of the simulation.

16. The system of claim 13, wherein the trajectories comprise an estimate of a force associated with the cutting instrument over a period of time.

17. The system of claim 16, wherein optimizing the set of parameters comprises applying stochastic gradient descent based on a set of ground-truth trajectories captured using sensors to measure real-world cutting operations.

18. A non-transitory computer readable medium storing instructions that, responsive to being executed by one or more processors, cause the one or more processors to:
receive a mesh for an object;
modify the mesh to add virtual nodes associated with a predefined cutting plane;
optimize a set of parameters associated with a simulator based on ground-truth data; and
run a simulation via the simulator to generate outputs that include trajectories associated with a cutting instrument.

19. The non-transitory computer-readable medium of claim 18, wherein running the simulation comprises, for each time step in a plurality of time steps:
calculating external contact forces between the object and a surface due to gravity;
calculating elastic forces for tetrahedral elements of the mesh based on a strain energy density formula;
calculating contact forces between the object and the cutting instrument;
updating the set of spring constants based on the contact forces; and
calculating spring forces associated with the virtual springs.

20. The non-transitory computer-readable medium of claim 17, wherein the trajectories comprise an estimate of a force associated with the cutting instrument over a period of time.
